SEQUENCE LISTING (1 sequence)

SEQ ID NO: 1 - protein, 21 aa, Homo sapiens
QDRLKFIDKQGELLAQDYKLR

(19) United States
(12) Patent Application Publication
(10) Pub. No.: US 2026/0191779 A1
(43) Pub. Date: Jul. 9, 2026

(54) Mitochondrial-Endoplasmic Reticulum Cell Death Inducing Nanoparticles

(71) Applicant: Northeastern University, Boston, MA (US)

(72) Inventors: Mansoor M. AMIJI, Attleboro, MA (US); Lara Scheherazade MILANE, Boston, MA (US)

(21) Appl. No.: 19/290,095

(22) Filed: Aug. 4, 2025

Related U.S. Application Data
(60) Provisional application No. 63/678,846, filed on Aug. 2, 2024.

(51) Int. Cl. A61K 9/127; A61K 31/427; A61K 31/706; A61K 38/16; A61P 35/00

(52) U.S. Cl. CPC A61K 9/127; A61K 31/427; A61K 31/706; A61K 38/16; A61P 35/00

(57) ABSTRACT

The present technology provides a combination approach for treating multidrug resistant cancer. Multidrug resistant cancers have more mitochondrial networks than drug sensitive cancers. A first agent fragments mitochondrial networks, dissociates mitochondria from the endoplasmic reticulum, and lower the threshold for apoptosis. A second active agent induces the unfolded protein response, causing stress to the endoplasmic reticulum and limiting the ability of multidrug resistant cancer cells to grow and survive. A third active agent directly activates mitochondrial apoptosis, leading to death of the cancer cells. The active agents can be combined into a nanoparticle formulation for simultaneous delivery into multidrug resistant cancer cells. The formulation serves as a nanomedicine for treatment of multidrug resistant cancers such as multidrug resistant triple negative breast cancer.

### CROSS REFERENCE TO RELATED APPLICATIONS

This application claims priority to U.S. Provisional Application No. 63/678,846 filed 2 Aug. 2024 which is incorporated by reference herein in its entirety.

### INCORPORATION BY REFERENCE STATEMENT

The content of the XML file of the sequence listing named “19815-1045-INV-25013-Sequence-Listing.xml”, having a size of 2053 bytes and a creation date of 9 Feb. 2026, and electronically submitted via Patent Center on 9 Feb. 2026, is incorporated herein by reference in its entirety.

### BACKGROUND

Multidrug resistant (MDR) cancer occurs when a cancer is not responsive to treatment. Multidrug resistant cancer occurs in all types of cancer and is responsible for the high mortality rate of cancer. Multidrug resistant cancer is very difficult to treat, as the cancer cells adapt in order to survive. The mitochondria of a cell are responsible for both energy production and programmed cell death. The endoplasmic reticulum (ER) produces all proteins in the cell. There are hundreds to thousands of mitochondria in a cell and one large ER network. Mitochondria are continually joining together in a network and breaking apart into individual structures; when mitochondria network, they can provide more energy to the cell and put their cell death function on hold. Mitochondria also can fuse with the ER; when this occurs, both energy production and protein synthesis are reduced.

The function of mitochondria and the ER, and modulation thereof by fusion of mitochondria with each other and with the ER, is particularly important to the survival of MDR cells, as these cells are constantly adapting so they can survive the challenges and drugs they encounter. Having a high energy supply and rate of protein synthesis are central to MDR cell survival.

There is a critical need for safer and more effective cancer treatments that attack MDR cells with reduced toxicity compared to the present standard of care.

### SUMMARY

The present technology provides compositions and methods for treating multidrug resistant (MDR) cancer cells, particularly mammalian cancer cells, and preferably human cells. The compositions and methods of the present technology incorporate nanoparticles each including at least one of a first agent which promotes the fragmentation of a mitochondrial-endoplasmic reticulum (ER) network within a cell; a second agent which activates the endoplasmic reticulum (ER) unfolded protein response in the cell; and a third agent, which activates apoptosis in the cell.

In one aspect, the technology provides a composition for treatment of a cancer, the composition comprising: a plurality of nanoparticles each comprising at least one agent for attacking a cancer cell; wherein the at least one agent attacks a cancer cell through at least one of: (a) fragmentation of a mitochondrial-endoplasmic reticulum network within the cancer cell; (b) activation of the endoplasmic reticulum unfolded protein response in the cancer cell; and (c) activation of apoptosis in the cancer cell.

As used herein, the term “anti-mitofusin” in used interchangeably with the terms “Anti-MFN” and/or “ANTI-MFN” and refers to an anti-mitofusin peptide including a peptide which corresponds to the cytoplasmic domain of the mitofusin peptide or includes an antibody or fragment thereof that binds to the mitofusin peptide.

As used herein, the term “anti-mitofusin 1” in used interchangeably with the terms “Anti-MFN1” and/or “ANTI-MFN1” and refers to an anti-mitofusin peptide including a peptide which corresponds to the cytoplasmic domain of the mitofusin 1 peptide or includes an antibody or fragment thereof that binds to the mitofusin 1 peptide.

As used herein, the term “anti-mitofusin 2” in used interchangeably with the terms “Anti-MFN2” and/or “ANTI-MFN2” and refers to an anti-mitofusin peptide including a peptide which corresponds to the cytoplasmic domain of the mitofusin 2 peptide or includes an antibody or fragment thereof that binds to the mitofusin 2 peptide.

As used herein the phrases “mitochondrial endoplasmic reticulum death inducing nanoparticle” “mitochondrial endoplasmic reticulum death inducing nanoparticles” are used are used interchangeably in respective singular and/or plural forms with terms “MEDI NP” and/or “MEDI NPs”.

As used herein, the phrases “mitochondrial network disrupting nanoparticle” and “mitochondrial network disrupting nanoparticles” are used are used interchangeably in respective singular and/or plural forms with the terms “MIND NP”, “MIND NP”, ‘MIND LNP”, and/or “MIND LNP” including poly(ethylene glycol)-modified (PEGylated), biocompatible liposome nanoparticle(s) each with an aqueous core encapsulating a validated anti-mitofusin 2 (Anti-MFN2) peptide fragment including amino acid sequence QDRLKFIDKQGELLAQDYKLR (SEQ ID NO:1).

The technology can be further summarized in the following list of features:
1. A composition for treatment of a multidrug resistant cancer, the composition comprising one or more, or two or more, of:
   a mitochondrial fragmentation agent;
   an unfolded protein response enhancing agent; and
   an apoptosis activating agent.
2. The composition of feature 1, wherein the composition comprises a mitochondrial fragmentation agent which is an anti-mitofusin peptide.
3. The composition of feature 2, wherein the anti-mitofusin peptide is an anti-mitofusin 2 peptide comprising SEQ ID NO:1 (QDRLKFIDKQGELLAQDYKLR), or a fragment or variant thereof having at least 70%, at least 80%, at least 90%, at least 95%, at least 98%, or at least 99% identity to SEQ ID NO:1.
4. The of composition of any of the preceding features, wherein the composition comprises an unfolded protein response enhancing agent which is tunicamycin.
5. The composition of any of the preceding features, wherein the composition comprises an apoptosis activating agent which is Bam7.
6. The composition of any of the preceding features, wherein one or more, or two or more, of said agents are packaged in a plurality of lipid nanoparticles or polymeric nanoparticles.
7. The composition of feature 6, wherein the composition comprises a plurality of lipid nanoparticles which are cationic liposomes.
8. The composition of feature 7, wherein the cationic liposomes comprise cationic lipid 1,2-dioleoyl-3-trimethylammonium-propane (DOTAP) and further comprise one or more components selected from the group consisting of neutral phospholipids, cholesterol, wherein one or more components of the liposomes is optionally conjugated to a hydrophilic polymer such as polyethylene glycol (PEG).
9. The composition of feature 7 or feature 8, wherein the cationic liposomes have a zeta potential greater than about +30 mV or less than about −30 mV.
10. The composition of feature 6, wherein the composition comprises a plurality of polymeric nanoparticles which are biodegradable polymeric nanoparticles.
11. The composition of feature 10, wherein the biodegradable polymeric nanoparticles comprise one or more biodegradable polymeric polymers selected from the group consisting of gelatin, chitosan, cellulose, cellulose derivatives, poly(ε-caprolactone) (PCL), polyethylene glycol (PEG), poly(lactic-co-glycolic acid) (PLGA), polylactic acid (PLA), polyglycolide, PEG-PLA diblock copolymer, PEG-PLGA diblock copolymer, PEG-PCL diblock copolymer, PCL-b-PEG-b-PCL co-polymer, polypropylene glycol (PPG), polyacrylic acid, polyacrylamide, poly(N-isopropylacrylamide), polyethylene oxide-polypropylene oxide-polyethylene oxide (PEO-PPO-PEO) triblock copolymer, hyaluronic acid, polyethylene oxide, polypropylene oxide, alginic acid, poly(betaaminoester) (PBAE), poly(glycolic acid) (PGA), and alginate.
12. The composition of any of features 6-11, wherein at least one of said agents is packaged in the lipid or polymeric nanoparticles, and at least one of said agents is not packaged in the lipid or polymeric nanoparticles.
13. The composition of feature 12, wherein the at least one of said agents that is not packaged in the lipid or polymeric nanoparticles is disposed in a liquid phase in which the lipid or polymeric nanoparticles are suspended.
14. The composition of any of features 6-13, wherein the nanoparticles are cationic liposomes and at least one of said agents is disposed within a lipid bilayer phase of the liposomes or within a lumen of the liposomes.
15. The composition of feature 14, wherein the cationic liposomes comprise said mitochondrial fragmentation agent disposed within the lumen of the liposomes, and/or wherein the cationic liposomes comprise said unfolded protein response enhancing agent disposed within the lipid bilayer phase of the liposomes, and/or wherein the cationic liposomes comprise said apoptosis activating agent within the lipid bilayer phase of the liposomes.
16. The composition of feature 15, wherein the cationic liposomes comprise an anti-mitofusin 2 peptide disposed within the lumen of the liposomes, and comprise tunicamycin and Bam7 disposed within the lipid bilayer phase of the liposomes.
17. The composition of any of the preceding features, wherein the composition further comprises an additional anti-cancer agent.
18. The composition of any of the preceding features, wherein the one or more active agents are packaged into a plurality of lipid nanoparticles or polymeric nanoparticles, and wherein the lipid nanoparticles or polymeric nanoparticles comprise a targeting agent that promotes selective delivery of said active agents to targeted cancer cells.
19. The composition of any of the preceding features, wherein the composition further comprises one or more excipients and is a pharmaceutical composition.
20. The composition of any of the preceding features, wherein administration of the composition to a mammalian subject in need thereof enhances death of multidrug resistant cancer cells within the subject.
21. A method of treating a multidrug resistant cancer in a mammalian subject in need thereof, the method comprising:
   (a) providing the composition of any of the preceding features; and
   (b) administering the composition to the mammalian subject, whereby death of multidrug resistant cancer cells of the mammalian subject is increased.
22. The method of feature 21, wherein, in cancer cells of the mammalian subject, fragmentation of mitochondria or a mitochondrial-endoplasmic reticulum network is enhanced, and/or wherein protein misfolding is enhanced, and/or wherein apoptosis is enhanced.
23. The method of feature 21 or feature 22, wherein one or more of the following is induced in cancer cells of the mammalian subject: decreased energy production, increased reactive oxygen species (ROS) production, increased nuclear DNA damage, increased mitochondrial DNA damage, decreased cell survival, decreased protein folding efficiency, increased protein misfolding, increased accumulation of misfolded proteins, decreased autophagy, decreased mitophagy, and/or increased apoptosis.
24. The method of any of features 21-23, wherein the mammalian subject is a human subject.
25. The method of any of features 21-24, wherein the multidrug resistant cancer is selected from the group consisting of solid tumors, blood cancers, breast cancer, ovarian cancer, lung cancer, and colorectal cancer.
26. A kit comprising the composition of any of features 1-20 and one or more additional components, such as one or more additional therapeutic agents for treatment of a cancer and/or instructions for carrying out the method of any of features 21-25.

### BRIEF DESCRIPTION OF THE DRAWINGS

FIG. 1A is a schematic illustration of an embodiment of a MEDI NP of the invention.

FIG. 1B is a schematic illustration of an embodiment of a MEDI NP including PEGylated and non-PEGylated anti-mitofusin 2 peptide, tunicamycin, and Bam7.

FIG. 2A demonstrates the hypoxic transformation including the nucleic translocation (activation) of HIF-1α in MDA-MB-231 and BT-549 cells after growth in hypoxic conditions for three and five days.

FIG. 2B demonstrates the hypoxic transformation in MDA-MB-231 and BT-549 cells including an increase in MDR1 expression after growth in hypoxic conditions for three and five days.

FIG. 2C shows total ATP production in normoxic and hypoxic MDA-MB-231 cells, and in a 50:50 co-culture of hypoxic and normoxic cells.

FIG. 3A shows fluorescent microscopy images of drug sensitive normoxic and multidrug resistant hypoxic MDA-MB-231 cells untreated and treated with mitochondrial network disrupting nanoparticles and includes images of mitochondria stained Mitotracker™ Green in a fluorescent channel obtained for mitochondrial network analysis.

FIG. 3B shows second images and overlays on first images of FIG. 3A for mitochondrial network analysis.

FIG. 3C shows the corresponding brightfield images and overlays of the first images of FIG. 3A for mitochondrial network analysis.

FIG. 3D shows converted binary images of FIG. 3A for mitochondrial network analysis.

FIG. 3E shows converted skeletonized images of FIG. 3A for mitochondrial network analysis.

FIG. 3F shows three-dimensional analyses based on FIGS. 3A-3E for mitochondrial network analysis.

FIG. 3G shows mitochondrial network quantification as a result of the analysis shown in FIG. 3F.

FIG. 4A shows fluorescent microscopy images of drug sensitive normoxic and multidrug resistant hypoxic BT-549 cells untreated and treated with mitochondrial network disrupting nanoparticles and includes images of mitochondria stained Mitotracker™ Green in a fluorescent channel obtained for mitochondrial network analysis (MiNA).

FIG. 4B shows second images and overlays on first images of FIG. 4A for mitochondrial network analysis.

FIG. 4C shows the corresponding brightfield images and overlays of the first images of FIG. 4A for mitochondrial network analysis.

FIG. 4D shows converted binary images of FIG. 4A for mitochondrial network analysis.

FIG. 4E shows converted skeletonized images of FIG. 4A for mitochondrial network analysis.

FIG. 4F shows three-dimensional analyses based on FIGS. 4A-44E for mitochondrial network analysis.

FIG. 4G shows mitochondrial network quantification as a result of the analysis shown in FIG. 4F.

FIG. 5A shows individual mitochondria per square micron for normoxic untreated cells (Norm Untreated 231), hypoxic untreated cells (Hyp Untreated 231), normoxic cells treated with mitochondrial network disrupting nanoparticles (MIND NPs) loaded with anti-MFN2 peptide (Norm MIND NP 231), hypoxic cells treated with mitochondrial network disrupting nanoparticles loaded with anti-MFN2 peptide (Hyp MIND NPs), normoxic cells treated with blank nanoparticles containing no peptide (Norm Blank NP 231), hypoxic cells treated with blank nanoparticles containing no peptide (Hyp Blank NP 231), normoxic cells treated with anti-MFN2 peptide in solution (Norm Sol 231), and hypoxic cells treated with anti-MFN2 peptide in solution (Hyp Sol 231).

FIG. 5B individual mitochondria per square micron for normoxic untreated cells (Norm Untreated 549), hypoxic untreated cells (Hyp Untreated 549), normoxic cells treated with mitochondrial network disrupting nanoparticles (MIND NPs) loaded with anti-MFN2 peptide (Norm MIND NP 549), hypoxic cells treated with mitochondrial network disrupting nanoparticles loaded with anti-MFN2 peptide (Hyp MIND NPs), normoxic cells treated with blank nanoparticles containing no peptide (Norm Blank NP 549), hypoxic cells treated with blank nanoparticles containing no peptide (Hyp Blank NP 549), normoxic cells treated with anti-MFN2 peptide in solution (Norm Sol 549), and hypoxic cells treated with anti-MFN2 peptide in solution (Hyp Sol 549).

FIG. 5C shows the mitochondrial networks per square micron for normoxic untreated cells (Norm Untreated 231), hypoxic untreated cells (Hyp Untreated 231), normoxic cells treated with mitochondrial network disrupting nanoparticles (MIND NPs) loaded with anti-MFN2 peptide (Norm MIND NP 231), hypoxic cells treated with mitochondrial network disrupting nanoparticles loaded with anti-MFN2 peptide (Hyp MIND NPs), normoxic cells treated with blank nanoparticles containing no peptide (Norm Blank NP 231), hypoxic cells treated with blank nanoparticles containing no peptide (Hyp Blank NP 231), normoxic cells treated with anti-MFN2 peptide in solution (Norm Sol 231), and hypoxic cells treated with anti-MFN2 peptide in solution (Hyp Sol 231).

FIG. 5D shows the mitochondria networks per square micron for normoxic untreated cells (Norm Untreated 549), hypoxic untreated cells (Hyp Untreated 549), normoxic cells treated with mitochondrial network disrupting nanoparticles (MIND NPs) loaded with anti-MFN2 peptide (Norm MIND NP 549), hypoxic cells treated with mitochondrial network disrupting nanoparticles loaded with anti-MFN2 peptide (Hyp MIND NPs), normoxic cells treated with blank nanoparticles containing no peptide (Norm Blank NP 549), hypoxic cells treated with blank nanoparticles containing no peptide (Hyp Blank NP 549), normoxic cells treated with anti-MFN2 peptide in solution (Norm Sol 549), and hypoxic cells treated with anti-MFN2 peptide in solution (Hyp Sol 549).

FIG. 5E shows the mitochondrial footprint per square micron for normoxic untreated cells (Norm Untreated 231), hypoxic untreated cells (Hyp Untreated 231), normoxic cells treated with mitochondrial network disrupting nanoparticles (MIND NPs) loaded with anti-MFN2 peptide (Norm MIND NP 231), hypoxic cells treated with mitochondrial network disrupting nanoparticles loaded with anti-MFN2 peptide (Hyp MIND NPs), normoxic cells treated with blank nanoparticles containing no peptide (Norm Blank NP 231), hypoxic cells treated with blank nanoparticles containing no peptide (Hyp Blank NP 231), normoxic cells treated with anti-MFN2 peptide in solution (Norm Sol 231), and hypoxic cells treated with anti-MFN2 peptide in solution (Hyp Sol 231).

FIG. 5F shows the mitochondrial footprint per square micron for normoxic untreated cells (Norm Untreated 549), hypoxic untreated cells (Hyp Untreated 549), normoxic cells treated with mitochondrial network disrupting nanoparticles (MIND NPs) loaded with anti-MFN2 peptide (Norm MIND NP 549), hypoxic cells treated with mitochondrial network disrupting nanoparticles loaded with anti-MFN2 peptide (Hyp MIND NPs), normoxic cells treated with blank nanoparticles containing no peptide (Norm Blank NP 549), hypoxic cells treated with blank nanoparticles containing no peptide (Hyp Blank NP 549), normoxic cells treated with anti-MFN2 peptide in solution (Norm Sol 549), and hypoxic cells treated with anti-MFN2 peptide in solution (Hyp Sol 549).

FIG. 6A shows % cell viability as a function of single agent drug treatment including each of Bam7, Tunicamycin, paclitaxel (PTX), mitochondrial network disrupting nanoparticles) (MIND NP) loaded with anti-MFN2 peptide, and blank nanoparticles containing no peptide (Blank NP) at 1 nM concentrations in normoxic and hypoxic MDA-MB-231 cells.

FIG. 6B shows % cell viability as a function of single agent drug treatment including each of Bam7, Tunicamycin, paclitaxel (PTX), mitochondrial network disrupting nanoparticles) (MIND NP) loaded with anti-MFN2 peptide, and blank nanoparticles containing no peptide (Blank NP) at 10 nM concentrations in normoxic and hypoxic MDA-MB-231 cells.

FIG. 6C shows % cell viability for single agent drug treatment including each of Bam7, Tunicamycin, paclitaxel (PTX), mitochondrial network disrupting nanoparticles) (MIND NP) loaded with anti-MFN2 peptide, and blank nanoparticles containing no peptide (Blank NP) at 001 nM concentrations in normoxic and hypoxic MDA-MB-231 cells.

FIG. 7A shows % cell viability as a function of the treatment of normoxic MDA-MB-231 cells with mitochondrial network disrupting nanoparticles (MIND NPs) loaded with anti-mitofusin 2 peptide; Bam7; a combination organelle mitochondrial-endoplasmic reticulum therapy (COMET) including triple combination of mitochondrial network disrupting nanoparticles (MIND NPs) loaded with anti-mitofusin 2 peptide, Bam7, and tunicamycin; and paclitaxel (PTX), at 1 uM, 10 nM, and 100 nM concentrations.

FIG. 7B shows % cell viability as a function of the treatment of hypoxic MDA-MB-231 cells with mitochondrial network disrupting nanoparticles (MIND NPs) loaded with anti-mitofusin 2 peptide; Bam7; a combination organelle mitochondrial-endoplasmic reticulum therapy (COMET) including triple combination of mitochondrial network disrupting nanoparticles (MIND NPs) loaded with anti-mitofusin 2 peptide, Bam7, and tunicamycin; and paclitaxel (PTX), at 1 uM, 10 nM, and 100 nM concentrations.

FIG. 8 shows % cell viability as a function of treatment with blank nanoparticles containing no peptide (Blank NP), 10 μM mitochondrial network disrupting nanoparticles (MIND NPs)) loaded with anti-mitofusin 2 peptide, 1 μM paclitaxel (PTX), and combination paclitaxel (PTX) (1 μM) and mitochondrial network disrupting nanoparticles (MIND NPs) (10 μM)) loaded with anti-mitofusin 2 peptide in hypoxic (Hyp) multidrug resistant cancer and normoxic (Norm) MDA-MB-231 cells.

FIG. 9 shows % cell viability as a function of treatment of human embryonic kidney epithelial cells (293T) mitochondrial network disrupting nanoparticles (MIND NPs) loaded with anti-mitofusin 2 peptide; Bam7; tunicamycin; blank nanoparticles containing no peptide (Blank NP); anti-MFN2 peptide in solution (Peptide Sol); a combination organelle mitochondrial-endoplasmic reticulum therapy (COMET) including triple combination of mitochondrial network disrupting nanoparticles (MIND NPs) loaded with anti-mitofusin 2 peptide, Bam7, and tunicamycin; and Paclitaxel (PTX),

FIG. 10 shows % complex V viability in complex V extracted from bovine mitochondria as a function of treatment with blank nanoparticles containing no peptide (Blank NP); anti-mitofusin 2 peptide solution (Anti-MFN Sol); mitochondrial network disrupting nanoparticles loaded with anti-mitofusin 2 peptide (Anti-MFN NP); and a solution of Rhodamine 6 G (R6G Sol), a specific complex V inhibitor used as a positive control.

FIG. 11A shows the lag time corresponding to the time between initiation of treatment and onset of activity in minutes as a function of treatment with mitochondrial network disrupting nanoparticles loaded with anti-mitofusin 2 peptide (Anti-MFN NP); blank nanoparticles containing no peptide (Blank NP); anti-mitofusin 2 peptide solution (Anti-MFN Sol); and a solution of Rhodamine 6 G (R6G Sol) in complex V extracted from bovine mitochondria.

FIG. 11B shows the time from the onset of activity and Tmax (time of maximal activity) as a function of treatment with mitochondrial network disrupting nanoparticles loaded with anti-mitofusin 2 peptide (Anti-MFN NP); blank nanoparticles containing no peptide (Blank NP); anti-mitofusin 2 peptide solution (Anti-MFN Sol); and a solution of Rhodamine 6 G (R6G Sol) in complex V extracted from bovine mitochondria.

FIG. 11C shows the Tmax (time of maximal activity) as a function of treatment with mitochondrial network disrupting nanoparticles loaded with anti-mitofusin 2 peptide (Anti-MFN NP); blank nanoparticles containing no peptide (Blank NP); anti-mitofusin 2 peptide solution (Anti-MFN Sol); and a solution of Rhodamine 6 G (R6G Sol) in complex V extracted from bovine mitochondria.

FIG. 12A shows the unfolded protein response (UPR) by the % change in normoxic MDA-MB-231 cells as a function of various treatments.

FIG. 12B shows the unfolded protein response (UPR) by the % change in hypoxic MDA-MB-231 cells as a function of various treatments.

FIG. 13A shows caspase 3 activity by the fold change relative to untreated cells in normoxic MDA-MB-231 cells as a function of various treatments.

FIG. 13B shows caspase 3 activity by fold change relative to untreated cells in normoxic MDA-MB-231 cells as a function of various treatments.

FIG. 14 shows the difference between hypoxic and normoxic caspase 3 induction by the fold change relative to untreated cells as a function of various treatments in hypoxic and normoxic MDA-MB-231 cells.

### DETAILED DESCRIPTION

The present technology provides new compositions, combination drug formulations, and methods for treatment of MDR cancer. The technology provides mitochondrial endoplasmic reticulum death inducing (MEDI) nanoparticles (NPs). The MEDI NPs are the first treatment for MDR cancer that manipulates organelle function and fusion.

The strategy of MEDI NP therapy is to manipulate the function and fusion of mitochondria and the endoplasmic reticulum. MEDI NPs can include at least one of three separate drugs or therapeutic agents that manipulate organelles in different ways, leading to death of MDR cancer cells, or enhancement of their susceptibility to other anti-cancer agents.

The first agent of MEDI NPs is a promoter of fragmentation the mitochondrial network in cells and also prevents mitochondria-to-endoplasmic reticulum fusion. Preventing fusion of mitochondria with each other and with the endoplasmic reticulum lowers the amount of energy the mitochondria can produce and restores the ability of mitochondria to induce programmed cell death, which makes treated cells more sensitive to drug challenges, and puts stress on the protein production of the endoplasmic reticulum.

The second agent functions to put even more stress on the endoplasmic reticulum by causing the cell to produce misfolded proteins and also increasing the sensitivity of the cell to death signaling.

The third agent directly activates the cell death function carried out by mitochondria. The MEDI NPs have high breakthrough potential, as the strategy of simultaneously manipulating organelle fusion and function has never been explored for treating multidrug resistant (MDR cancer). The MEDI NPs can be as effective yet significantly less toxic compared to standard of care chemotherapy in cell models.

Cancer cells have an elevated apoptotic threshold resulting in resistance to cell death. This apoptotic threshold is even higher in multidrug resistant (MDR) cancer cells. Multidrug resistance (MDR) is a significant clinical obstacle for all cancers, contributing to recurrent disease. Functioning as the central mediators of apoptosis and energy production, mitochondria are critical to apoptotic resistance in cancer cells. Mitochondria continually fuse together to form networks and break apart or undergo fission (mitochondrial dynamics). Mitofusin (MFN) proteins (e.g., mitofusin 1 (MFN1) or mitofusin 2 (MFN2) mediate mitochondrial fusion. Mitochondria fused into network conformation are resistant to apoptosis. Mitochondrial fission forms scission sites on the outer mitochondrial membrane). Pro-apoptotic signaling leads to Bax (bcl-2-like protein 4) association with mitofusin proteins such as mitofusin 1 and/or mitofusin 2 at the outer mitochondrial membrane scission sites, initiating apoptosis. Mitochondria also fuse with the endoplasmic reticulum through mitofusin 1 and/or mitofusin 2 mediation. Mitochondrial-to-endoplasmic reticulum fusion directly couples mitochondrial energy production to endoplasmic reticulum protein synthesis, enabling the high protein synthesis capacity of cancer cells. Mitochondrial fusion to the endoplasmic reticulum also rescues cells during the endoplasmic reticulum unfolded protein response and promotes cell survival over cell death.

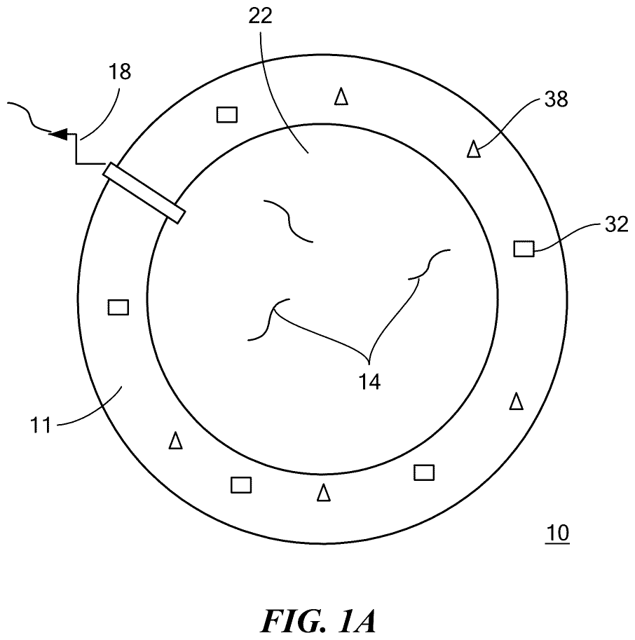

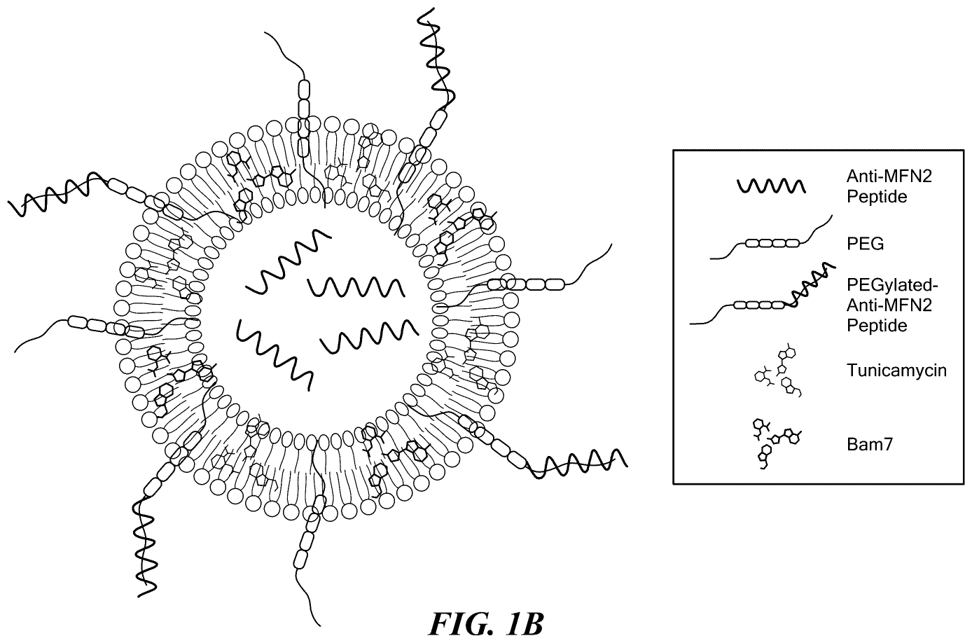

The present technology includes mitochondrial endoplasmic reticulum death inducing nanoparticles (MEDI NPs) 10 shown schematically shown in FIG. 1A for directly targeting this biological axis of multidrug resistant cancer. Due to the unique manipulation of critical organelle processes and the use of a nanoparticle formulation, the MEDI NPs offer greater efficacy and safety in treating multidrug resistant cancer in comparison to standard chemotherapy as shown using in vitro models.

The mitochondrial endoplasmic reticulum death inducing nanoparticles MEDI NPs 10 include nanoparticles such as liposomes 12 that contain anti-mitofusin peptide including anti-mitofusin 1 (Anti-MFN1) and/or anti-mitofusin 2 (Anti-MFN2) peptide and preferably an anti-MFN2 peptide. The anti-mitofusin peptide includes a peptide which corresponds to the cytoplasmic domain of human mitofusin or includes an antibody or fragment thereof that binds to the mitofusin peptide such as mitofusin-1 or mitofusin-2.

Variations in the amino acid sequence of anti-MFN2 can include, for example, 1, 2, 3, 4, or 5 conservative amino acid substitutions. Conservative amino acid substitutions in a peptide or polypeptide are substitutions of an amino acid with an equivalent amino acid which do not substantially alter the structure and/or functionality of the peptide or polypeptide. Equivalent amino acids have side chains with similar properties such as bulkiness, polarity (polar or non-polar), hydrophobicity (hydrophobic or hydrophilic), pH-dependent protonatable groups (acidic, neutral or basic), and organization of carbon molecules (aromatic or aliphatic).

Amino acids can be divided into the following classes, within which amino acids are considered equivalent. Substitutions made within the same class are considered conservative substitutions.
1. Amino acids having polar side chains (Asp, Glu, Lys, Arg, His, Asn, Gln, Ser, Thr, Tyr, and Cys)
2. Amino acids having non-polar side chains (Gly, Ala, Val, Leu, Ile, Phe, Trp, Pro, and Met)
3. Amino acids having aliphatic side chains (Gly, Ala Val, Leu, Ile)
1. Amino acids having cyclic side chains (Phe, Tyr, Trp, His, Pro)
2. Amino acids having aromatic side chains (Phe, Tyr, Trp)
3. Amino acids having acidic side chains (Asp, Glu)
4. Amino acids having basic side chains (Lys, Arg, His)
5. Amino acids having amide side chains (Asn, Gln)
6. Amino acids having hydroxy side chains (Ser, Thr)
7. Amino acids having sulfur-containing side chains (Cys, Met),
8. Neutral, weakly hydrophobic amino acids (Pro, Ala, Gly, Ser, Thr)
9. Hydrophilic, acidic amino acids (Gln, Asn, Glu, Asp), and
10. Hydrophobic amino acids (Leu, Ile, Val)

An active agent can be administered, for example as packaged in lipid- or polymer-based nanoparticles, in its active, mature form or in a precursor form that is released in the subject's body or in the target cell to provide the active form.

Also preferred are fragments or variants of SEQ ID NO:1, such as: (i) fragments or variants of SEQ ID NO:1 having at least 70%, at least 80%, at least 90%, at least 95%, at least 98%, or at least 99% identity to SEQ ID NO: 1; fragments of SEQ ID NO: 1 containing at least 5, at least 6, at least 7, at least 8, at least 9, at least 10, at least 11, at least 12, at least 13, at least 14, or at least 15 consecutive amino acids of SEQ ID NO: 1; and variants of SEQ ID NO: 1 containing from 1 to 10, from 1 to 5, or from 1 to 3 conservative amino acid substitutions compared to either SEQ ID NO:1.

Compositions of the present technology can be pharmaceutical compositions, comprising one or more active agents and one or more excipients of any kind. Excipients can include, for example, diluents, buffers, carriers, adjuvants, salts, and the like. While pharmaceutical compositions can take many forms, such as liquids, suspensions, solids, tablets, powders, capsules, or aerosols, a preferred form is an aqueous suspension of nanoparticles formulated for parenteral administration, such as intravenous or subcutaneous injection, or direct injection into a tumor or another tissue. Also contemplated are solid forms that release nanoparticles of the present technology, such as over a period of time, into surrounding tissue, or into the circulatory system, the lymphatic system, or the central nervous system. Nanoparticles of the present technology, optionally embedded in a biodegradable polymer matrix, can be implanted anywhere in the subject's body for subsequent release. Implants can be used as part of or in conjunction with a medical device, including an implantable medical device. U.S. Pat. No. 12,121,608 (hereby incorporated by reference in its entirety) describes an implant for delivery through the olfactory epithelium into the brain, bypassing the blood-brain barrier; such an implant can be used to deliver nanoparticles of the present technology to the brain or other parts of the central nervous system. Certain pH-sensitive biodegradable polymers, such as Eudragit (a copolymer of methyl methacrylate and/or other alkyl methacrylates), also can be used for oral delivery of any of the active agents described herein; see, for example, U.S. Pat. No. 11,491,114, which is hereby incorporated by reference in its entirety.

Pharmaceutical compositions for use in the present technology include a plurality of nanoparticles that contain at least two active agents: an inhibitor of extracellular vesicle release and an inhibitor of tunneling nanotube formation. In a preferred embodiment, the nanoparticles contain both types of active agent; more preferably each nanoparticle contains both types of active agent. Also contemplated are pharmaceutical compositions containing a mixture of two or more different populations of nanoparticles, each population containing one or more active agents that are different from the active agent(s) contained in other populations of nanoparticles. Preferably, the nanoparticles are suspended in an aqueous medium, which is preferably isotonic and suitable for injection into the subject's body. In alternative embodiments, the nanoparticles can be suspended in a solid, gel, or other formulation for administration to the subject by implantation, oral delivery, transmucosal delivery, or topical application.

FIG. 1A schematically illustrates a liposome embodiment of a MEDI NP (10) according to the present technology. The liposome possesses lipid bilayer membrane 11 surrounding aqueous lumen 22. Hydrophilic active agent 14 (e.g., anti-mitofusin 2 peptide) can be encapsulated within the lumen, or conjugated to a lipid or PEGylated lipid component of the liposome, as shown in 18. Hydrophobic active agents such as 32 (e.g., tunicamycin) and 38 (e.g., Bam7) can be embedded within the lipid bilayer. FIG. 1B provides another schematic illustration if a MEDI NP configured as a liposome.

When MEDI NPs are taken up by cancer cells, especially multidrug resistant cancer cells, the NPs are broken down and the active agents released into the cell. For example, anti-mitofusin 2 peptide or an anti-mitofusin 2 antibody can be released from the core of the NPs. Similar agents conjugated to PEGylated acyl chains or lipid molecules protrude from the surface of the NPs and can be released by enzymatic cleavage or other NP breakdown processes, and then freed to bind to and inactivate mitofusin 2 on mitochondria, leading to increased fragmentation of the cell's mitochondrial networks and reduction of mitochondria-to-endoplasmic reticulum fusion; this lowers the apoptotic threshold, lowers oxidative phosphorylation capacity, and sensitizes the ER to stress. Agents that increase the unfolded protein response (32) such as tunicamycin can be released from the lipid bilayer of the MEDI NPs, and then can induce the ER unfolded protein response, thereby sensitizing the cancer cells to death by other agents. The unfolded protein response can lead to a cellular decision point: cell death or survival. Without mitochondrial rescue from the unfolded protein response, the stress in multidrug resistant cells such as triple negative breast cancer cells is more likely to induce cell death. Agents that activate apoptosis (38) such as Bam7, an activator of Bax (bcl-2-like protein 4) also can be released from the lipid bilayer of the MEDI NPs, thereby directly activating Bax (bcl-2-like protein 4) to initiate intrinsic apoptosis. Bax translocates to the mitochondrial outer membrane and initiates apoptosis through the release of cytochrome C, apoptosome formation, and caspases 3 induced cell death. If any two or all three of such agents are included, the action of each of the agents potentiates the action of the other agents, producing a powerful killing effect of multidrug resistant cancer cells.

The chemical structures for tunicamycin and Bam7 are shown below.

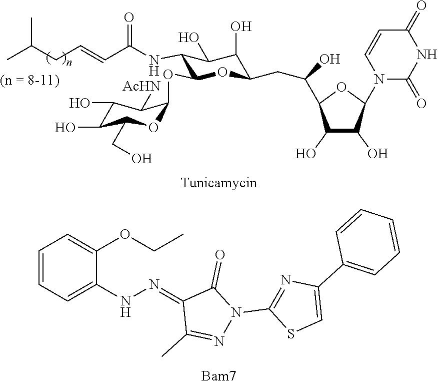

Studies described in the examples below of the MEDI NPs have demonstrated that the MEDI NPs are as effective as the standard of care in treating multidrug resistant cancer including triple negative breast cancer cells, yet are significantly less toxic to normal cells.

MEDI NPs are the first treatment for multidrug resistant cancer that manipulates mitochondrial and endoplasmic reticulum function and fusion. The mitochondrial endoplasmic reticulum death inducing nanoparticles MEDI NPs have the potential to transform the treatment of multidrug resistant cancer. The breaking apart of mitochondrial networks and the prevention of mitochondrial-to-endoplasmic reticulum fusion before inducing the unfolded protein response and the activation of intrinsic apoptosis can revolutionize the therapy of multidrug resistant cancer.

The present technology provides compositions, combination drug formulations, kits, and methods which have advantages over previous technologies for treating multidrug resistant cancer. The advantages include: improved efficacy over the standard of care; improved safety over the standard of care; the direct targeting of the underlying biology of multidrug resistant cancer; and direct manipulation of multidrug resistant cancer cells at the organelle level.

The combination organelle mitochondrial endoplasmic reticulum therapy thus involves a possible three-prong approach to treat multidrug resistant cancers such as multidrug resistant triple negative breast cancer (TNBC). In the first prong, mitochondrial networks are fragmented with mitochondrial network disrupting (MIND) nanoparticles (NPs). This fragmentation lowers the apoptotic threshold of cells and sensitizes multidrug resistant cancer cells to death in three ways (1) networked mitochondria are conformationally resistant to pro-apoptotic signaling, (2) fragmented mitochondria have a lower capacity for oxidative phosphorylation, and (3) disassociating mitochondria from the endoplasmic reticulum lowers the capacity for high rates of protein synthesis and lowers the ability of mitochondria to rescue the endoplasmic reticulum from stress. After mitochondrial networks are fragmented, in the second prong, combination organelle mitochondrial endoplasmic reticulum therapy uses an agent such as tunicamycin to induce the unfolded protein response in the endoplasmic reticulum, further lowering the apoptotic threshold. Lastly, in the third prong, combination organelle mitochondrial endoplasmic reticulum therapy uses an agent such as Bam7, a direct pro-apoptotic Bax (bcl-2-like protein 4) activator to directly initiate mitochondrial apoptosis. The combination organelle mitochondrial endoplasmic reticulum therapy is a promising early-stage translational nanomedicine for treating multidrug resistant cancers such as multidrug resistant triple negative breast cancer (TNBC). The combination organelle mitochondrial endoplasmic reticulum therapy exploits the fusion and function of mitochondria and the endoplasmic reticulum and elevates the concept that mitochondria are so much more than just the powerhouse of the cell.

The present technology presents the design, development, and evaluation of combination organelle mitochondrial endoplasmic reticulum therapy (COMET) as a novel nanomedicine for multidrug resistant cancers such as multidrug resistant triple negative breast cancer (TNBC). The combination organelle mitochondrial endoplasmic reticulum therapy (COMET) successively manipulates the function and fusion of mitochondria and the endoplasmic reticulum to orchestrate cell death in multidrug resistant cancer disease.

The therapeutic rationale of combination organelle mitochondrial endoplasmic reticulum therapy (COMET) addresses a key principle of cell biology; there is perpetual connection and communication between all parts of the cell, including organelles. Mitochondria are not lonely, peanut-shaped organelles hovering in the off skirts of the cell. Mitochondria are responsive. Mitochondria continually fuse in a network and fiss apart^{1,2}, like a large school of fish making their way across the Mediterranean Sea together. The school of fish may network together for protection while mitochondria fused in networks are oriented in an anti-apoptotic conformation (more resistant to cell death) that aids cell survival. The school of fish may migrate across the sea seeking warmer temperatures or a replenished food supply while mitochondria will network and fiss (iterative process to move) across a cell to supply energy to a demanding process such as movement or protein synthesis. The school of fish may come together to maintain their species and evolve, similar to mitochondrial repair through mitophagy and mitophagic triggering of autophagy.

Cellular organelles are interactive and continuous. The endoplasmic reticulum is continuous with the nuclear envelope and forms contact sites with microtubules and the plasma membrane^{3,4}; mitochondria fuse with each other and with the endoplasmic reticulum (ER)^{1,2} creating an intertwined continuum of organelles. Mitochondrial fusion to the endoplasmic reticulum (ER) directly couples energy supply to the energy demanding process of protein synthesis. This axis of fused organelles is critical to cell survival and is an underexplored target for cancer therapeutics that combination organelle mitochondrial endoplasmic reticulum therapy (COMET) directly manipulates.

Cancer is the leading cause of death in the world while breast cancer specifically is the fifth leading cause of death globally and the second leading cause of cancer death in women in the United States^{5,6}. Triple negative breast cancer (TNBC) is a highly drug resistant and metastatic form of breast cancer that accounts for up to 20% of all breast cancer cases and predominantly effects younger women, black women, and women with the BRCA1 mutation⁷. Triple negative breast cancer is negative for the estrogen and progesterone receptors as well as the human epidermal growth factor receptor 2 (HER2) protein⁷. The National Cancer Institute has identified that drug resistance remains “one of the most challenging problems facing cancer researchers and patients today” 8,9. This is particularly significant for patients with triple negative breast cancer (TNBC) as there are limited treatments and the rate of recurrent disease that is multidrug resistant is very high, leading to poor prognosis⁷. When the 13 hallmarks of cancer¹⁰⁻¹² are considered, cancer may seem like an extremely complex and complicated disease with hallmarks ranging from tumor promoting inflammation to deregulated cellular energetics. Yet there is one single hallmark that summarizes the biology of the disease and this is cellular plasticity¹⁰. Cancer is simply survival of the fittest on a cellular level; cancer is a disease of accelerated molecular evolution where a cell will adapt and change however necessary in order to survive in a perpetually changing microenvironment¹⁰. Multidrug resistant cancer is a trait that develops as cancer cells evolve to survive local challenges such as nutrient and oxygen deprivation. At the time of diagnosis, some cancers present as innately drug resistant meaning they have already acquired the mechanisms of drug resistance before any drug exposure^{7,13-15}. Conversely, some cancers acquire multidrug resistance after initiating drug treatment^{7,13-15}. Both innate and acquired multidrug resistance are a challenge to the clinical management of almost all cancers; multidrug resistant cancer is not an anomaly, multidrug resistant cancer is the clinical norm^{7,13-15}.

Mitochondrial dysfunction is central to multidrug resistance cancers as mitochondria are the mediators of cell death and energy production, both of which are altered in cancer^{16,17}. Mitochondrial network fusion is mediated by the mitofusin proteins 1 (MFN1) and 2 (MFN2), with a predominance of mitofusin protein 2 (MFN2) mediation^{18,19}. Mitochondrial fusion increases oxidative phosphorylation (OXPHOS) capacityl⁹⁻²². Mitochondrial network fusion also protects cells from death¹⁹⁻²². Mitochondrial fission is required for intrinsic apoptosis¹⁹⁻²³. Mitochondrial fission enables pro-apoptotic Bcl-2 family members (Bax corresponding to bcl-2-like protein 4) to bind to the mitochondrial outer membrane (OMM) and begin the apoptotic cascade^{22,24-27}. During fission, foci are formed on the outer mitochondrial membrane (OMM); Bax (bcl-2-like protein 4) binds to these foci and associates with mitofusin protein 2 (MFN2) at the scission sites²⁴⁻²⁷.

Mitofusin 2 (MFN2) also mediates mitochondrial fusion with the endoplasmic reticulum (ER). The endoplasmic reticulum (ER) is responsible for protein synthesis and folding; as cancer cells are continually adapting and evolving to ensure their survival in perpetually changing tumor microenvironmental conditions, there is a constant demand for high levels of protein synthesis²⁸⁻³⁰. Mitochondrial fusion to the endoplasmic reticulum (ER) directly couples energy supply to the energy demanding process of protein synthesis, enabling cancer cells to continue to survive challenges through responsive alterations in protein expression³¹⁻³⁵.

A second implication of mitochondrial-endoplasmic reticulum (ER) fusion is mitochondrial rescue of the endoplasmic reticulum (ER) from the unfolded protein response (UPR)^{31,36-39}. The unfolded protein response (UPR) is a scripted response that can lead to cell death or cell survival⁴⁰. Endoplasmic reticulum (ER) stress increases mitochondrial-endoplasmic reticulum (ER) fusion, Ca²⁺ exchange, and oxidative phosphorylation (OXPHOS)⁴¹. In cancer, the unfolded protein response (UPR) can trigger autophagy and lead to cellular repair while mitochondrial-endoplasmic reticulum (ER) Ca²⁺ exchange increases metabolism and survival⁴².

The therapeutic strategy of combination organelle mitochondrial endoplasmic reticulum therapy (COMET) is illustrated in FIGS. 1F-1G, as discussed above. First, mitochondrial network disrupting nanoparticles (MiND NPs) fragment critical mitochondrial networks and prevent mitochondrial fusion to the endoplasmic reticulum (ER). The mitochondrial network disrupting nanoparticles (MIND NPs) contain a first agent such as a validated anti-mitofusin 2 (MFN2) peptide²⁰. This lowers the apoptotic threshold, decreases oxidative phosphorylation (OXPHOS), and sensitizes cells to stress. The mitochondrial network disrupting nanoparticles (MIND NPs) contain a second agent such as tunicamycin for induction of endoplasmic reticulum (ER) stress which leads to the unfolded protein response (UPR)^{39,43}. Without mitochondrial rescue, the unfolded protein response (UPR) favors cell death over survival. The mitochondrial network disrupting nanoparticles (MIND NPs) contain a third agent such as Bam7 for activation of the proapoptotic Bcl-2 family member, Bax (bcl-2-like protein 4), directly initiates mitochondrial apoptosis⁴⁴. The present technology demonstrates that mitochondrial network disrupting nanoparticles (MIND NPs) lower the apoptotic threshold of multidrug resistant cancer cells such as multidrug resistant triple negative breast cancer (MDR TNBC) cells and decrease oxidative phosphorylation (OXPHOS) while tunicamycin induces the unfolded protein response (UPR). The present technology demonstrates that combination organelle mitochondrial endoplasmic reticulum therapy (COMET) is as effective as paclitaxel (standard of care) with significantly lower toxicity to normal cells in vitro.

The treatment of multidrug resistant cancer such as multidrug resistant triple negative breast cancer (MDR TNBC) requires simultaneous, neoadjuvant, or adjuvant combination therapy as drugs with different mechanisms of action for overcoming the multidrug resistance^{45,46}. Newer, safer, more effective treatments that target the molecular biology of multidrug resistant cancer ( ) are needed. To decrease toxicity, combination organelle mitochondrial endoplasmic reticulum therapy (COMET) employs nanoparticles to deliver the anti-mitofusin (MFN) peptide. The nanoparticles (NPs) can also be used to deliver a second agent such as tunicamycin and a third agent such as Bam7, for increasing the safety and tumor specificity of treatment^{17,47-52}.

The nanoparticles including liposomes of the present technology have strong translational potential and are very similar to the lipid nanoparticles (NPs) used in the current Pfizer (BNT162b2) and Moderna (mRNA1273) SARS-COV-2 vaccines. Like these mRNA vaccines, lipid nanoparticles enable the delivery of agents such as RNA and peptides that would otherwise be degraded before reaching their molecular target and lipid nanoparticles have a high safety profile, even after repeated dosing regimens^{49,53-61}. A second safety advantage of nanoparticle (NP) liposomal delivery is related to the large surface area to volume of the lipid nanoparticles (NPs) which protect a patient from high systemic free drug concentrations while protecting the drug from immune clearance^{17,49,62}. This advantage allows for a lower effective drug administration as the drug is more highly protected from immune and metabolic degradation while the lower effective dose results in lower toxicity. The third advantage of the lipid nanoparticle (NP) approach is the enhanced permeability and retention (EPR) effect of lipid nanoparticles (NPs) which results in preferential solid tumor accumulation^{52,63,64}. Enhanced permeability and retention (EPR) results from the lipid nanoparticles (NPs) traversing the leaky vasculature of tumors (more than the intact vasculature of normal tissue) and the prolonged retention of lipid nanoparticles (NPs) in the tumor due to poor lymphatic drainage^{52,63,64}.

The combination organelle mitochondrial endoplasmic reticulum therapy (COMET) is designed as a translational nanomedicine with the intent of clinical translation and is intended as a first line therapy for triple negative breast cancer (TNBC) and multidrug resistant triple negative breast cancer (MDR TNBC) to achieve disease-free survival. Current first line therapy for triple negative breast cancer (TNBC) includes a combination of radiation and chemotherapy or a two to three drug sequential monotherapy regimen with prolonged drug exposure and substantial toxicity⁶⁵. This toxicity often requires treatment modification and/or drug-free intervals that can further promote acquired multi drug resistance⁶⁵. The combination organelle mitochondrial endoplasmic reticulum therapy (COMET) is designed as a neoadjuvant combination intravenous or i.v. therapy; with mitochondrial network disrupting nanoparticles (MIND NPs) administered prior to the co-administration of tunicamycin and Bam7. The employment of a nanomedicine approach has enabled the combination organelle mitochondrial endoplasmic reticulum therapy (COMET) to have less toxicity than the standard of care in vitro. The use of the combination organelle mitochondrial endoplasmic reticulum therapy (COMET) as the first treatment for multidrug resistant cancer such as multidrug resistant triple negative breast cancer (MDR TNBC) for the manipulation of mitochondrial and endoplasmic reticulum function and fusion is an advancement in molecular therapeutics with strong potential for reduced toxicity in vivo.

### Nanoparticles

The nanoparticles of the present technology can be used as delivery vehicles to transport an active agent to its target cell in a variety of contexts. The nanoparticles can be introduced into the blood or lymphatic system or injected directly into the interstitial fluid of an organ or tissue, for example. In some cases, however, the nanoparticles can be administered so as to overcome or cross a biological barrier, such as the blood-brain barrier, the intestinal mucosal barrier, a basement membrane, or an epithelial cell layer. In such cases, administering the nanoparticles across the biological barrier can be achieved, for example, by penetrating the barrier with a device to perform injection across the barrier, or by implantation of a time-release composition or device that releases the nanoparticles directly across the barrier or into a target organ or tissue. Alternatively, the size or other properties of the nanoparticles can be selected so that the nanoparticles are able to cross the biological barrier. For example, small nanoparticles having a size of about 50 nm or less may be able to cross the biological barrier without further intervention.

The nanoparticles including mitochondrial endoplasmic reticulum death inducing nanoparticles MEDI NPs include lipid nanoparticles or biodegradable polymeric nanoparticles, wherein the lipid nanoparticles or biodegradable polymeric nanoparticles encapsulate at least one of three agents.

As used herein, a “biodegradable polymeric nanoparticle” includes a polymer susceptible to degradation (or biodegradation) after implantation into an organism, wherein the degradation is accompanied by lowering of the polymer's molar mass. The biodegradation can proceed, for example, by hydrolysis, by contact with nasal mucus, by catalytic activity of other enzymes, or by a combination of factors including a wide variety of biological activities. The support body encloses or contains a reservoir, i.e., an open space that can be filled with a polymer matrix, such as a hydrogel. The support body can include openings and/or pores connecting the reservoir with the environment outside the support body. The reservoir can be filled with a polymer network or matrix that contains a second biodegradable polymer which forms the polymer matrix.

The biodegradable polymeric nanoparticles disclosed herein are capable of controlled rates of degradation, for example, by selecting different copolymers, by changing pore size, by selecting a thickness of the polymer, or by utilizing layers (e.g., core-shell) of polymers of varying thicknesses. When a polymer is utilized as an outer shell or hollow sheath, the term “biodegrades” can refer to cracking of an outer shell.

In an example, a hydrophilic or hydrophobic outer sheath biodegradable polymeric nanoparticle can include pores and a core-shell configuration, wherein the polymeric outer sheath around the core acts as a barrier layer to delay and further control the release profile of a therapeutic agent. The size of the pores can be adjusted, for example, by utilizing different molecular weights of polyethylene glycol (PEG) with poly(ε-caprolactone) in an outer sheath polymer. The pores can allow diffusion of the at least one of three agents through the outer sheath polymer to contact the olfactory mucosal epithelium. Pores can increase or decrease in size after implantation. The size of the pores can be in the range from about 0.01 μm to about 100 μm, in the range from about 1 μm to about 100 μm, in the range from about 5 μm to about 50 μm, or in the range from about 10 μm to about 30 μm. In an example, an outer sheath polymer can have an average wall thickness in the range from about 0.1 mm to about 2 mm, or in the range from about 0.1 mm to about 1 mm, or in the range from about 0.1 mm to about 0.6 mm.

The at least one agent for attacking a cancer cell can be provided as dissolved or suspended within the biodegradable polymeric nanoparticles as solid particles, such as nanoparticles or microparticles, within a hydrogel. The hydrogel can be an osmotic hydrogel that contains an osmotic core component, such as a dissolved or suspended osmolyte that attracts water from the biological environment to form a water-swollen polymer network that promotes the release of the at least one agent for attacking a cancer cell from the gel. The hydrogel can include a polymer network, a colloidal network, or a combination thereof. The hydrogel can encapsulate the payload (e.g., at least one agent for attacking a cancer cell and modulate its release profile by hydrolytic swelling. Hydrogels are highly hydrophilic networks of polymer chains, sometimes found as colloidal gel networks in which water is the dispersion medium. The water-absorbing properties of the hydrogels can result from the presence of hydrophilic functional groups, for example, hydroxy (—OH), carboxylic (—COOH), amidic (—CONH—), primary amidic (—CONH2), and sulfonic (—SO3H), rather than from the osmotic pressure of the hydrogels. In hydrogel swelling, examples of relevant parameters are the swelling rate, swelling ratio, and swelling capacity, which can depend on several physiochemical factors such as the gel size, network porosity, network structure, cross-linking conditions, and cross-linking degree of the hydrogel. While typically not considered in higher molecular weight formulations, the osmolarity of hydrogels can also contribute to swelling or water uptake. The normal osmolarity of the human body is in the range from about 250 to about 350 milliosmoles. Optionally, the osmolarity of the hydrogel can be adjusted to change, for example, the swelling rate.

The biodegradable polymeric nanoparticles of the present technology include one or more biodegradable polymers selected from the group consisting of gelatin, chitosan, cellulose, cellulose derivatives, poly(ε-caprolactone) (PCL), polyethylene glycol (PEG), poly(lactic-co-glycolic acid) (PLGA), polylactic acid (PLA), polyglycolide, PEG-PLA diblock copolymer, PEG-PLGA diblock copolymer, PEG-PCL diblock copolymer, PCL-b-PEG-b-PCL co-polymer, polypropylene glycol (PPG), polyacrylic acid, polyacrylamide, poly(N-isopropylacrylamide), polyethylene oxide-polypropylene oxide-polyethylene oxide (PEO-PPO-PEO) triblock copolymer, hyaluronic acid, polyethylene oxide, polypropylene oxide, alginic acid, poly(betaaminoester) (PBAE), poly(glycolic acid) (PGA), and alginate.

In selected embodiments, the nanoparticles including the at least one agent for attacking a cancer cell include lipid nanoparticles including preferably liposomes, and more preferably cationic liposomes.

Cationic lipids are used in the present technology to promote cellular uptake of the at least one agent for attacking a cancer cell. Cationic lipids can be incorporated into liposomes, forming cationic liposomes, or can form non-membranous lipid complexes with one or more agents thereby forming mixed micelles or other supramolecular structures. Liposomes and other lipid-containing complexes are referred to herein as “lipid nanoparticles”. Inclusion of cationic lipids in such lipid nanoparticles can promote the stabilization of an agent for delivery into a cell via endocytosis, especially if the agent is negatively charged. Cationic lipids also promote endosomal escape, thereby releasing the agent into the cytoplasm.

Any type of cationic lipid can be used in the present technology. Cationic lipids typically have a positively charged headgroup and a hydrophobic tail, and can be phospholipids or other types of lipids. Examples of cationic lipids include dioctadecyltrimethylammonium (DOTMA), dioctadecyltetramethylammonium (DOTAP), and dimethylaminoethane carbamoyl cholesterol. Various other lipids can be combined with cationic lipids in order to provide lipid nanoparticles with desired properties, as is known in the field.

Cationic liposomes can be drawn to or target the negative potential of the cellular mitochondria. The cationic liposomes include 1,2-dioleoyl-3-trimethylammonium-propane (DOTAP) as the cationic lipid for facilitating preferential mitochondrial association and further include one or more components selected from the group consisting of a neutral phospholipid for bilayer formation, cholesterol for structural integrity, and a hydrophilic polymer such as polyethylene glycol (PEG) for decreasing particle aggregation and decreasing immune clearance. In one embodiment, the composition consists of 44 wt. % dipalmitoylphosphatidylcholine (DPPC), 14 wt. % cholesterol 22 wt. % 1,2-dioleoyl-3-trimethylammonium-propane (DOTAP); and 20% polyethylene glycol (PEG).

In preferred embodiments, the zeta potential of the liposomes is greater than about +30 mV or less than about −30 mV. In alternative embodiments, the zeta potential of the liposomes is in a range from about 0 mV to about +100 mV, or from about 0 mV to about −100 mV, or from about +30 mV to about +100 mV, or from about −30 mV to about −100 mV, or from about +40 mV to about +60 mV, or from about −40 mV to about −60 mV. Preferably, the zeta potential is in a range that promotes stability and avoids aggregation of the liposomes.

The cationic liposomes are prepared by forming a lipid film, rehydrating the film with the peptide solution in water during freeze thaw cycles to form multilaminar vesicles, and probe sonicating to form liposomes. The lipid film is prepared using a cationic lipid such as DOTAP (1,2-dioleoyl-3-trimethylammonium-propane chloride salt), a stabilizer such as cholesterol, and a neutral lipid such as DPPC (1,2-dipalmitoyl-sn-7 glycero-3-phosphocholine), in a 5:3:5 molar ratio in 2 ml of chloroform using a Sigma-Aldrich ST/NS14/20 10 mL round bottom flask attached to a Rotavap (IKA works Inc. Wilmington, NC-28405, Model RV 10 C S99). Following chloroform evaporation, the films are resuspended in 1 ml of 1 mg peptide solution in di water before 5 cycles of liquid nitrogen freezing and heating in a 42C water bath. The liposomal preparation is then probe-sonicated for 5 min on ice. The mixture is centrifuged in a Beckman-Coulter ultracentrifuge at 20,000 RPM for 15 min in Amicon (10K) centrifugal filters at 4 C to separate the peptide-encapsulated liposomes from unencapsulated peptide.

In selected embodiments, the compositions include one or more pharmaceutically acceptable excipients or carriers. The pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to a subject. Water, saline solutions and aqueous dextrose and glycerol solutions can also be employed as liquid excipients. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents.

The compositions of the present technology can be present in various formulations. Any compound and composition (and/or additional agents) described herein can take the form of solutions, suspensions, emulsion, drops, tablets, pills, pellets, capsules, capsules containing liquids, powders, sustained-release formulations, or any other form suitable for use. In one embodiment, the composition is in the form of a capsule (see, e.g., U.S. Pat. No. 5,698,155). Other examples of suitable pharmaceutical excipients are described in Remington's Pharmaceutical Sciences 1447-1676 (Alfonso R. Gennaro eds., 19th ed. 1995), incorporated herein by reference.

Where necessary, the compounds and compositions can also include a solubilizing agent. Also, the compounds and compositions can be delivered with a suitable vehicle or delivery device as known in the art. Combination therapies outlined herein can be co-delivered in a single delivery vehicle or delivery device.

The formulations comprising the compounds and compositions of the present invention may conveniently be presented in unit dosage forms and may be prepared by any of the methods well known in the art of pharmacy. Such methods generally include the step of bringing the therapeutic agents into association with a carrier, which constitutes one or more accessory ingredients. Typically, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

Routes of administration include, for example: intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin.

In some embodiments, the composition is administered orally. In some embodiments, the administration is by absorption through epithelial or mucocutaneous linings (e.g., oral mucosa). Various delivery systems are known, e.g., encapsulation in liposomes, microparticles, microcapsules, capsules, etc., and can be used to administer. In various embodiments, the compounds and compositions of the present invention are formulated to be suitable for oral delivery. In various embodiments, the compounds and compositions of the present invention are formulated to be suitable for transmucosal delivery (see, e.g. Msatheesh, et al. Expert Opin Drug Deliv. 2012 June; 9 (6): 629-47, the entire contents of which are hereby incorporated by reference).

Compositions or compounds for oral delivery can be in the form of tablets, lozenges, aqueous or oily suspensions, granules, powders, emulsions, capsules, syrups, or elixirs, for example. In some embodiments, the compounds and compositions of the present invention are in the form of a capsule, tablet, patch, or lozenge. Orally administered compositions can comprise one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active capsule containing a driving compound capable of driving any compound or composition described herein is also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be useful. Oral compositions can include standard excipients such as mannitol, lactose, starch, magnesium stearate, sodium saccharin, cellulose, and magnesium carbonate. In one embodiment, the excipients are of pharmaceutical grade. Suspensions, in addition to the active compounds, may contain suspending agents such as, for example, ethoxylated isostearyl alcohols, polyoxyethylene sorbitol and sorbitan esters, microcrystalline cellulose, aluminum metahydroxide, bentonite, agar-agar, tragacanth, etc., and mixtures thereof.

### EXAMPLES

### Example 1. Materials and Methods

### Nanoparticle Synthesis and Characterization

Mitochondrial network disrupting liposome nanoparticles (MIND NPs) were prepared similar to published methods⁶⁶. Lipid films were prepared using 1,2-dipalmitoyl-sn-7 glycero-3-phosphocholine (DPPC) as neutral lipid; 1,2-dioleoyl-3-trimethylammonium-propane chloride salt (DOTAP) as cationic lipid; cholesterol as stabilizer, and polyethylene glycol (PEG-200) in the w/w % ratios listed in Table 1. For formation of the films, a stock concentration of each lipid was made in chloroform and 1 mL of each lipid was added to a Sigma-Aldrich ST/NS14/20 10 mL round bottom flask attached to a Rotavap (IKA works Inc. Wilmington, NC-28405, Model RV 10 C S99), and allowed to rotate at 100 rpm in a water bath at room temperature. After film formation, the flask was dried for 24 hours in a vacuum desiccating chamber.

For mitochondrial network disruption, a validated anti-MFN2 peptide²⁰ having the sequence QDRLKFIDKQGELLAQDYKLR (SEQ ID NO:1) was incorporated into the core of the liposomes. This was accomplished by rehydrating the lipid film with 1 mg of peptide in deionized water. For blank liposomes, the lipid films were rehydrated with 1 ml of deionized water. For formation of the vesicles, the rehydrated films were passed through five freeze-thaw-vortex cycles. Liquid nitrogen was used for freezing. The Rotavap (with no vacuum) was used for thawing. The water bath was set at 42° C. which is above the transition temperature of the lipids. For formation of smaller liposomes, the preparation was probe sonicated for five minutes on ice and subsequently centrifuged Beckman-Coulter ultracentrifuge at 20,000 revolutions per minute (RPM) for 15 minutes at 4° C. to collect liposomes in membrane filter tubes.

### Cell Culture, Induction of Multidrug Resistance (MDR), and Cell Viability

Cell lines from the NCI-60 panel, which corresponds to a panel of 60 human cancer cell lines used by the National Cancer Institute (NCI) for screening compounds to identify potential anticancer activity, were selected due to their established application in the screening of cancer drug efficacy. The two triple negative breast cancer (TNBC) lines selected from this panel were purchased from ATCC®; MDA-MB-231 and BT-549 cells. Human embryonic kidney epithelial cells (293T) were used for toxicity analysis and were also purchased from ATCC®.

Cells were maintained at 37° C. in Dulbecco's Modified Eagle Medium (DMEM), a basal cell culture medium that supports the growth of many mammalian cells supplemented with 10% fetal bovine serum and 1% penicillin/streptomycin/amphotericin B. For creation of multidrug resistant derivatives of each cell line, a modular incubation chamber (Billups-Rothenberg, Inc.; Del Mar, CA) was flushed with a 0.5% O₂, 5% CO₂, nitrogen balanced gas at a rate of 20 liters/minute for five minutes after continuous flow in the chamber and incubated at 37° C. Hypoxia treatment for all experiments was 5-days to ensure multidrug resistant transformation.

For microscopy, cells were seeded at a density of 100,000 cells in 1.5 ml media in μ-Dish 35 mm high Glass Bottom dishes (Ibidi®). Following 2-hour treatments with drugs and nanoparticles, mitochondria were stained with 250 nM Mitotracker™ Green FM (ThermoFischer) for 45 minutes. Before imaging, dye loaded media was replaced with complete media.

For cell viability studies, cells were seeded at 2,000 cells per well and treated with drugs, drug combinations, and nanoparticles for 5 days. Wells with media alone (no cells) were used as the experimental background, while wells with cells alone were the negative control, and cells treated with 10% triton-X were used as a positive control for cell death. Cell viability was measured using Promega's 3-(4,5-dimethylthiazol-2-yl)-5-(3-carboxymethoxyphenyl)-2-(4-sulfophenyl)-2H-tetrazolium (MTS) assay and n=24 for each experimental treatment.

### Enzyme-Linked Immunosorbent Assay (ELISA)

Basal and nucleic protein was extracted from cells after 90% confluency was reached in 25 cm² tissue culture flasks. Radioimmunoprecipitation Assay (RIPA) high salt buffer was used for extraction of basal protein, and nucleic protein was extracted using a Nuclear and Cytoplasmic Extraction Reagent (NE-PER™) extraction kit (ThermoFisher). The Bicinchoninic Acid (BCA) protein assay was used to quantify protein using the microplate procedure (ThermoFisher). Hypoxia-inducible factor (HIF)-1α (MBS261767) and multidrug resistance 1 (MDR1) (MBS9428612) enzyme-linked immunosorbent assay (ELISA) kits (MyBioSource.com) were used to quantify nucleic hypoxia-inducible factor (HIF) and basal multidrug resistance 1 (MDR1) via sandwich enzyme-linked immunosorbent assay (ELISA).

### Microscopy and Mitochondrial Network Analysis

A Keyence BZ-X710 fluorescence microscope was used to acquire images for mitochondrial network analysis (MiNA). Mitochondrial network analysis (MiNA) software⁶⁷ was used to quantify mitochondrial networks in normoxic, hypoxic, and treated cells. The mitochondrial network analysis (MiNA) involved a series of image processing steps of the fluorescent channel. The steps include unsharpening the mask, enhancing local contrast, filtering the median, converting to binary, skeletonizing the image, analyzing the 2D/3D skeleton, and quantifying the network parameters⁶⁷. The mitochondrial network analysis (MiNA) outputs three-dimensional (3D) quantification of mitochondrial networks, individual mitochondria, mean branch length, median branch length, length standard deviation, mean network size (branches), median network size (branches), network size standard deviation, and the mitochondrial footprint⁶⁷. The mitochondrial footprint is the number of pixels with signal in the binary image multiplied by the total area of a pixel⁶⁷. The footprint accounts for mitochondria area not mathematically identified by the exclusion criteria of the software as being in a network or fragmented. For each experimental condition and cell line an n=25 was used.

### Oxidative Phosphorylatio, the Unfolded Protein Response, Adenosine Triphosphate, and Caspase 3 Activity

Human mitochondrial synthase, often denoted as complex V, is one of the five complexes known to be involved in oxidative phosphorylation (OXPHOS). This complex phosphorylates adenosine diphosphate (ADP) to adenosine triphosphate (ATP) with the help of energy generated by the proton electrochemical gradient. It comprises of two functional domains—F0 (located in the inner mitochondrial membrane) and F1 (located in the mitochondrial matrix). The testing of mitochondrial function, especially the changes in the activity of complex V after treatment with liposomes, evaluates if there is a direct inhibitory effect of anti-mitofusin 2 liposomes on complex V. MitoTox™ Complex V OXPHOS activity assay kit (Abcam; ab109907) was used.

Complex V was extracted from bovine heart mitochondria and was immunocaptured by anti-complex V monoclonal antibody on the plate. A dose response method was performed and results were obtained using a plate reader (OD—340 nm).

The effect of the combination organelle mitochondrial endoplasmic reticulum therapy (COMET) for altering the oxidative phosphorylation (OXPHOS) capacity of cells through mitochondrial network changes was determined with a measurement of total adenosine triphosphate (ATP). Promega's Mitochondrial ToxGlo™ Assay was used to quantify ATP in normoxic and hypoxic cells after different treatment conditions using a thermostable Ultra-Glo™ luciferase reporter. The unfolded protein response (UPR) was measured using Montana Molecular's green, fluorescent cell stress sensor kit (WO2019014072A1). The assay used a fluorescence biosensor that detected splicing of the XBP1 RNA mediated by upstream ER stress proteins. Caspase 3 is an effector caspase of both the intrinsic and extrinsic apoptotic pathway. Abcam's colorimetric caspase 3 kit (ab39401) was used to quantify caspase 3 in normoxic and hypoxic cells after treatment. The assay detected caspase sequence specific binding to the chromophore p-nitroaniline.

### Example 1: Mitochondrial Network Disrupting Nanoparticle (MIND NP) Characterization

The MIND NPs of the present technology include biocompatible liposomes; lipid bilayer particles with an aqueous compartment for anti-mitofusin peptide delivery. These liposomes have strong translational potential and are very similar to the lipid nanoparticles used in the current Pfizer (BNT162b2) and Moderna (mRNA1273) SARS-COV-2 vaccines.

Nanoparticles were prepared as either blank nanoparticles loaded no peptide (Blank NP) or MIND NPs loaded with an anti-MFN2 peptide. A Malvern Pan Analytical Zeta Sizer was used to measure nanoparticle size (via dynamic light scattering) and zeta potential. A Nanodrop One spectrophotometer was used to determine the encapsulation efficiency. N=20.

Table 1 demonstrates that the mitochondrial network disrupting nanoparticles (MIND NP) of the present technology have a reproducible liposome size (164 nm±21), stability (zeta potential+42 mV±5), and peptide encapsulation (73%±8). The blank nanoparticles were loaded with no peptide (Blank NP) but are otherwise similar in composition and structure to the peptide loaded MIND NP. The liposomal NPs are ideal for treating cancer due to enhanced permeability across leaky tumor vasculature (relative to normal tissue) and increased retention in tumors with poor lymphatic drainage.

TABLE 1
Mitochondrial Network Disrupting (MIND)
Nanoparticle (NP) Characterization

| | Size | Zeta | |
|---|---|---|---|
| | (d.nm) | Potential | % E.E. |
| Blank NP | 188 nm ± 34 | +39 mV ± 7 | — |
| MIND NP | 164 nm ± 21 | +42 mV ± 5 | 73% ± 8 |

| | |
|---|---|
| Composition | 44.12% DPPC, 13.92% Cholesterol, |
| (w/w %): | 32.34% DOTAP, 9.62% PEG-2000 |
| Anti- | QDRLKFIDKQGELLAQDYKLR |
| mitofusin 2 | |
| peptide: | |

The anti-mitofusin 2 peptide is derived from the heptad repeat region (HR1) of mitofusin 2²⁰. Due to the presence of charged, hydrophilic, and hydrophobic residues, the HR1 region is capable of ionic interactions with the internal HR2 region of mitofusin 2, the HR1/HR2 regions of other mitofusin proteins, and capable of amphiphilic insertion into the outer mitochondrial membrane of neighboring mitochondria upon fusion^{18,68-71}. The anti-mitofusin 2 peptide has a grand average of hydropathy (GRAVY) index of −1.08, indicating that the peptide is hydrophilic and has a net charge of +1 at physiological pH.

For validation of the ionic interaction of the anti-mitofusin 2 peptide with mitofusin 2, two different scramble sequences of the anti-mitofusin 2 peptide with the same GRAVY score and charge were created. The anti-mitofusin 2 peptides were encapsulated in liposome nanoparticles, and each demonstrated comparable mitochondrial network fragmentation (not shown).

The present examples use liposome nanoparticles for the anti-MFN2 peptide delivery while tunicamycin and Bam7 were administered in solution.

### Example 2: Hypoxic Modeling of Multidrug Resistance

Hypoxia is a well-established driver of multidrug resistant cancer in cancer⁷²⁻⁸¹. Solid tumors undergo perpetual microenvironmental changes including vasculature remodeling (angiogenesis and vascular destruction). These changes lead to regions of transient and sustained hypoxia. Under conditions of hypoxia, Hypoxia Inducible Factor-1α (HIF-1α) translocates from the cytoplasm to the nucleus and complexes with Hypoxia Inducible Factor-β (HIF-β) to form an active transcription factor^{72,73}. HIF target genes include genes that contribute to multidrug resistant cancer (including multidrug resistance protein 1 (MRP1); multidrug resistance 1 (MDR1)), proliferation, and survival under nutrient and oxygen deprivation^{72,73}. Hypoxia has also been shown to promote cancer survival by inducing metabolic changes including an increase in and aerobic glycolysis known as the Warburg effect and both reported increases and decreases in oxidative phosphorylation^{72,73,77,82,83} Although there are multiple Hypoxia HIF-α isoforms, HIF-1α is the predominant isoform⁸⁴.

A validated hypoxic conditioning method was used^{75,76,79,85-92} to create multidrug resistant derivatives of the two triple negative breast cancer (TNBC) cell lines. The biological basis of this method was the hypoxic induction of multidrug resistance through activation of HIF-1α transcription factor^{72,73}.

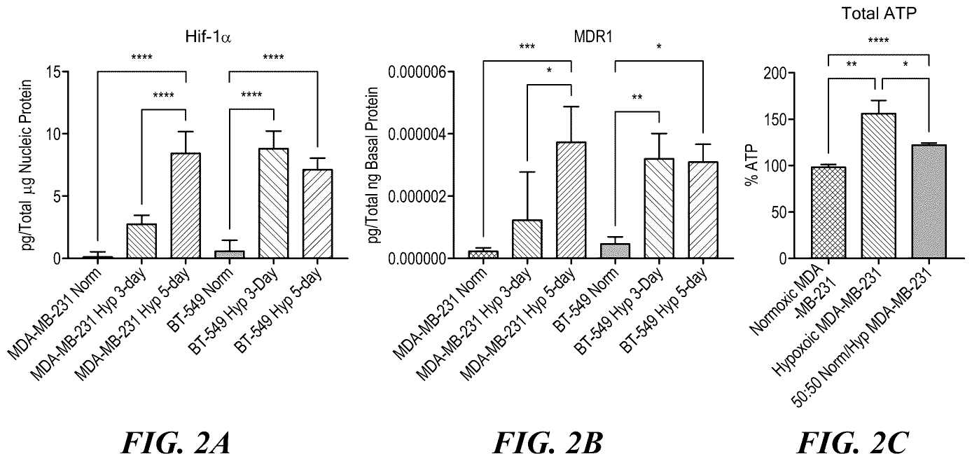

FIGS. 2A-2C demonstrate hypoxia induced nucleic translocation of HIF1-α which leads to MDR1 upregulation, increased ATP production, and increased paclitaxel (PTX) resistance. ELISA of nucleic (A) and basal (B) proteins demonstrated that hypoxic induction of MDR1 in two TNBC cell lines (MDA-MB-231 and BT-549) occurred between 3-5 days of hypoxic exposure. Significance was only shown within each cell line and was determined using a Welch's t-test. Translocation of HIF-1α to the nucleus (FIG. 2A) corresponds to upregulation of the drug efflux pump multidrug resistance 1 (MDR1) (FIG. 2B). FIG. 2C demonstrates that hypoxia also transforms the energetics (total ATP production) of MDA-MB-231 cells and this transformation can be passed from hypoxic cells to normoxic cells through a mix of 50% hypoxic cells and 50% normoxic cells. The effect of 5 days hypoxia on PTX half-maximal inhibitory concentration (IC₅₀) values in the two cell lines was evaluated as PTX is an MDR1 substrate (Table 2D). Hypoxia resulted in a 70.67 fold change in half-maximal inhibitory concentration IC₅₀) values for MDA-MB-231 cells and a 32.62 fold change in the BT-549 cells. The increased difference in hypoxic PTX half-maximal inhibitory concentration (IC₅₀) values in the MDA-MB-231 cells corresponds to a greater significant increase in MDR1 expression in hypoxic verses normoxic cells in comparison to the BT-549 cells.

TABLE 2D

| Cell Line & Condition | PTX IC₅₀(μM) | Fold change |
|---|---|---|
| MDA-MB-231 Normoxic | 0.015 ± 0.03 | 70.67 |
| MDA-MB-231 Hypoxic | 1.06 ± 0.31 | |
| BT-549 Normoxic | 0.042 ± 0.19 | 32.62 |
| BT-549 Hypoxic | 1.37 ± 0.48 | |

Thus, FIGS. 2A-2C and Table 2D demonstrate the hypoxic transformation in MDA-MB-231 and BT-549 cells after growth in hypoxic conditions for three and five days. FIG. 2A illustrates that three days of hypoxia is sufficient to significantly increase the nucleic translocation (activation) of HIF-1α in BT-549 cells. The MDA-MB-231 cells require five days of growth in hypoxic conditions for significant activation of HIF-1α relative to normoxic (Norm) conditions.

FIG. 2B validates that nucleic translocation of HIF-1α is coupled to an increase in MDR1 expression. Three days of hypoxia was sufficient to increase both nucleic HIF-1α translocation and basal MDR1 in the BT-549 cells. Five days of hypoxia was required for a significant increase of nucleic HIF-1α and basal MDR1 in MDA-MB-231 cells. The increase of MDR1 expression after five days of hypoxia was more significant (relative to normoxic growth) in the MDA-MB-231 than in the BT-549 cells.

The metabolic differences between normoxic and hypoxic cells were also evaluated by comparing total ATP levels in MDA-MB-231 cells (FIG. 2C). Five days of hypoxia significantly increased the total ATP production of the cells and this transformation could be passed from hypoxic cells to normoxic cells (50:50 co-culture). Equal numbers of normoxic cells and cells grown in hypoxia for five days were mixed in a co-culture and total ATP was measured after two days of co-incubation. The co-culture (column 3, FIG. 2C) ATP levels were more similar to the hypoxic MDA-MB-231 cells than to the normoxic cells indicating either that the hypoxic cells could transfer their increased energy capacity to normoxic cells or that the hypoxic induced increase in energy capacity could not be sufficiently diluted by normoxic cells.

For further validation of the hypoxic induction of MDR, a dose response study of the normoxic and hypoxic cells to PTX was performed to calculate half-maximal inhibitory concentration (IC₅₀) values (Table 2D). Hypoxia significantly increased the half-maximal inhibitory concentration (IC₅₀) of PTX in both cell lines, indicating an increase in resistance to PTX induced cell death. PTX was selected as it is an MDR1 substrate. Hypoxia had a more pronounced effect on the PTX half-maximal inhibitory concentration (IC₅₀) in the MDA-MB-231 cells which also corresponds to a more significant increase of hypoxia induced MDR1 (Panel B) in this cell line.

### Example 3: Mitochondrial Network Analysis

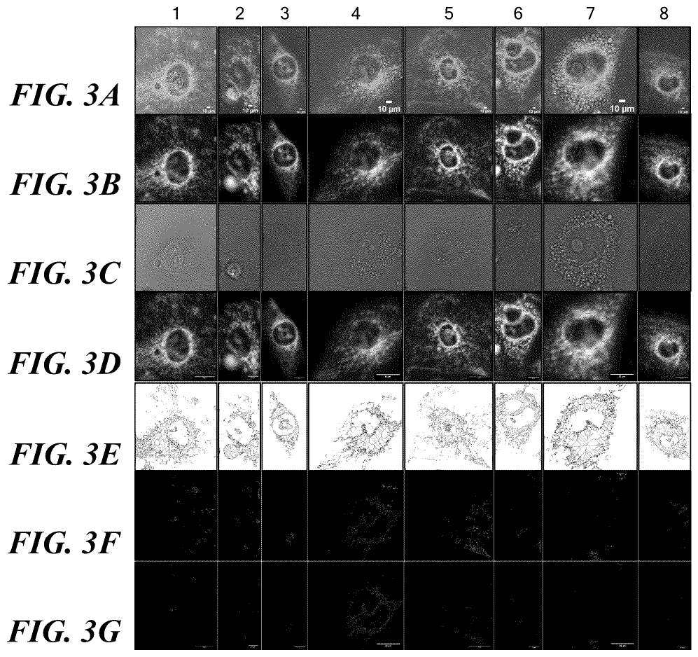

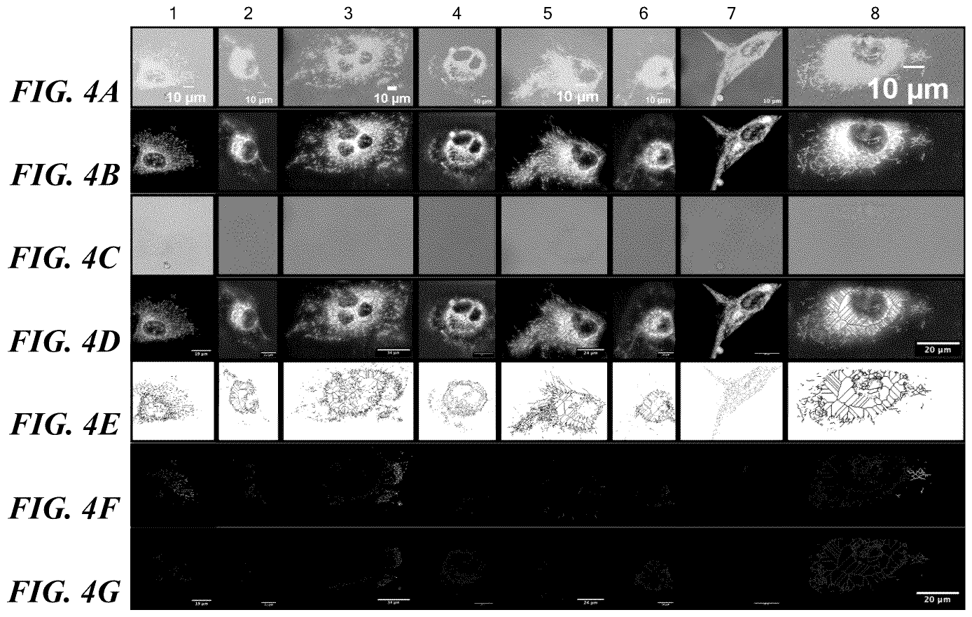

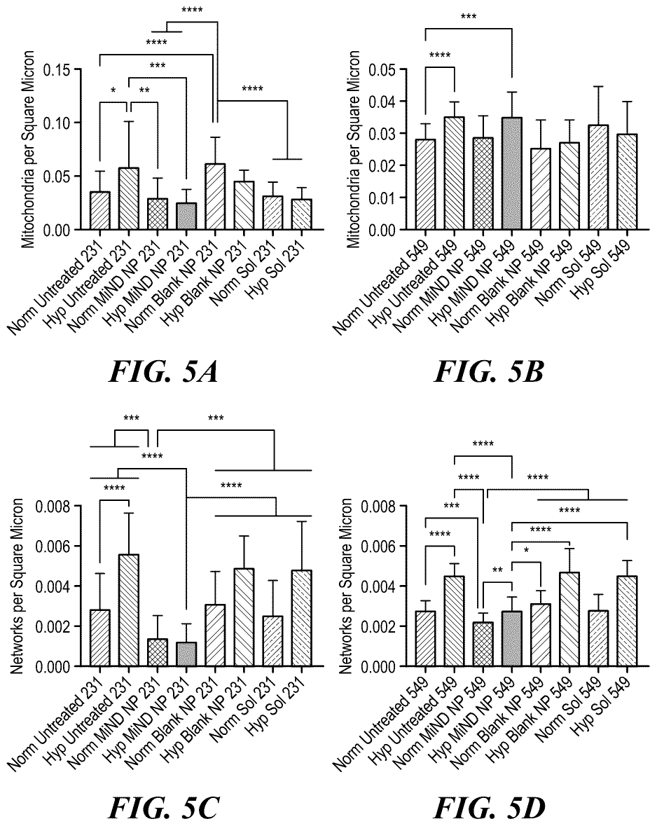

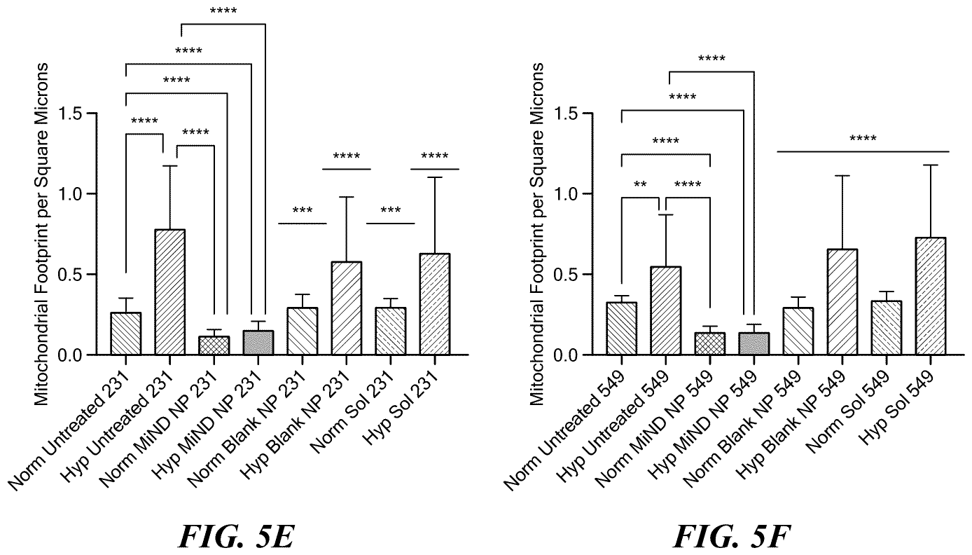

Mitochondrial Network Analysis (MiNA) was used to quantify hypoxia and treatment induced changes in individual mitochondria and mitochondrial networks was central to this study. Fluorescent microscopy images of MDA-MB-231 cells (FIGS. 3A-G) and BT-549 cells (FIGS. 4A-G) were used for the MiNA (FIGS. 5A-F). MiNA is a tool for measuring mitochondrial dynamics⁶⁷. Alternative methods for measuring mitochondrial dynamics rely on methods that do not mimic endogenous events. The most common method for assessing mitochondrial fusion is to use polyethylene glycol to fuse two cell populations with differentially labeled (red and green) mitochondria and observe co-localization of the labeling⁹³. The result of this method is the creation of a hybrid cell and this technique is not actually evaluating the mitochondrial dynamics within one cell, but rather assessing the fusion of mitochondria as the result of two cells fusing together.

In contrast to such alternative methods, mathematical expressions from MiNA were used to calculate mitochondrial network parameters (FIGS. 5A-F) from three-dimensional (3D) imaging data (FIGS. 3A-G and FIGS. 4A-G)⁶⁷. Due to differences in cell sizes, all MiNA data was normalized to the square micron area. FIGS. 3A-G and FIGS. 4A-G are representative images from a n=25 for each condition in each cell line. The columns in FIGS. 3A-G and FIGS. 4A-G are representative of the different treatment groups, namely, (1) untreated normoxic cells, (2) normoxic cells treated with MIND NPs, (3) normoxic blank NPs, (4) normoxic peptide solution, (5) untreated hypoxic cells, (6) hypoxic cells treated with MIND NPs, (7) hypoxic blank NPs, and (8) hypoxic peptide solution. The rows corresponding to FIGS. 3A-G and FIGS. 4A-G are the MiNA steps where respective FIGS. 3A and 4A represents an overlay of the green fluorescent channel (FIGS. 3B and 4B) and the brightfield image (FIGS. 3C and 4C). FIGS. 3D-G and 4D-G show the MiNA processing of the stained mitochondrial skeleton (FIGS. 3D and 4D), binary conversion (FIGS. 3E and 4E), two-dimensional (2D) network analysis (FIGS. 3F and 4F), and three-dimensional (3D) network quantification (FIGS. 3G and 4G).

FIGS. 3A-G show microscopy and mitochondrial network analysis of untreated and MIND nanoparticle treated drug sensitive and multidrug resistant MDA-MB-231 cells. Normoxic and hypoxic (multidrug resistant cancer) MDA-MB-231 cells were stained with 250 nM Mitotracker™ Green and fluorescent microscopy was performed using the 60× objective of a Keyence BZ-X710 microscope. Images were processed using MiNA. The scale bars on the FIG. 3A overlay are 10 μm. The columns correspond to (1) Untreated Normoxic Cells, (2) Normoxic Cells Treated with Mitochondrial Network Disrupting Nanoparticles (MIND NPs), (3) Normoxic Blank Nanoparticles (NPs), (4) Normoxic Peptide Solution, (5) Untreated Hypoxic Cells, (6) Hypoxic Cells Treated with Mitochondrial Network Disrupting Nanoparticles (MIND NPs), (7) Hypoxic Blank Nanoparticles (NPs), and (8) Hypoxic Peptide Solution. FIG. 3A represents an overlay of the green fluorescent channel (FIG. 3B) and the brightfield image (FIG. 3C). FIGS. 3D-G are mitochondrial network analysis (MiNA) processing of the stained mitochondrial skeleton (FIG. 3D), binary conversion (FIG. 3E), 2D network analysis (FIG. 3F), and 3D network quantification (FIG. 3G).

FIGS. 4A-4F shows microscopy and mitochondrial network analysis of untreated and mitochondrial network disrupting (MIND) nanoparticle treated drug sensitive and multidrug resistant BT-549 cells. Normoxic and Hypoxic (multidrug resistant cancer) BT-549 cells were stained with 250 nM Mitotracker™ Green and fluorescent microscopy was performed using the 60× objective of a Keyence BZ-X710 microscope. Images were processed using mitochondrial network analysis (MiNA). The scale bars on the panel A overlay are 10 μm. Columns: (1) Untreated Normoxic Cells, (2) Normoxic Cells Treated with Mitochondrial Network Disrupting Nanoparticles (MIND NPs), (3) Normoxic Blank Nanoparticles (NPs), (4) Normoxic Peptide Solution, (5) Untreated Hypoxic Cells, (6) Hypoxic Cells Treated with Mitochondrial Network Disrupting Nanoparticles (MIND NPs), (7) Hypoxic Blank Nanoparticles (NPs), and (8) Hypoxic Peptide Solution. FIG. 4A represents an overlay of the green fluorescent channel (FIG. 4B) and the brightfield image (FIG. 4C). Figures D-G are mitochondrial network analysis (MiNA) processing of the stained mitochondrial skeleton (FIG. 4D), binary conversion (FIG. 4E), 2D network analysis (FIG. 4F), and 3D network quantification (FIG. 4G).

FIGS. 5A, 5C and 5E shows mitochondrial network analysis (MiNA) of MDA-MB-231 cells including mitochondria per square micron for normoxic untreated cells (Norm Untreated 231), hypoxic untreated cells (Hyp Untreated 231), normoxic cells treated with mitochondrial network disrupting nanoparticles (MIND NPs) loaded with anti-MFN2 peptide (Norm MIND NP 231), hypoxic cells treated with mitochondrial network disrupting nanoparticles loaded with anti-MFN2 peptide (Hyp MIND NPs), normoxic cells treated with blank nanoparticles containing no peptide (Norm Blank NP 231), hypoxic cells treated with blank nanoparticles containing no peptide (Hyp Blank NP 231), normoxic cells treated with anti-MFN2 peptide in solution (Norm Sol 231), and hypoxic cells treated with anti-MFN2 peptide in solution (Hyp Sol 231).

FIGS. 5B, 5D and 5F shows mitochondrial network analysis (MiNA) of BT-549 cells including mitochondria per square micron for normoxic untreated cells (Norm Untreated 549), hypoxic untreated cells (Hyp Untreated 549), normoxic cells treated with mitochondrial network disrupting nanoparticles (MIND NPs) loaded with anti-MFN2 peptide (Norm MIND NP 549), hypoxic cells treated with mitochondrial network disrupting nanoparticles loaded with anti-MFN2 peptide (Hyp MIND NPs), normoxic cells treated with blank nanoparticles containing no peptide (Norm Blank NP 549), hypoxic cells treated with blank nanoparticles containing no peptide (Hyp Blank NP 549), normoxic cells treated with anti-MFN2 peptide in solution (Norm Sol 549), and hypoxic cells treated with anti-MFN2 peptide in solution (Hyp Sol 549).

FIGS. 5A-F show that hypoxic induced multidrug resistance increases mitochondrial networks while mitochondrial network disrupting nanoparticles (MIND NPs) fragment mitochondrial networks in MDA-MB-231 and BT-549 TNBC cells. Mitochondrial network analysis (MiNA) of 25 samples per group was performed to quantify changes induced by hypoxia and with mitochondrial network disrupting nanoparticle (MIND NP) treatment in MDA-MB-231 cells (FIGS. 5A, 5C, 5E) and in BT-549 cells (5B, 5D, 5F). Hypoxia (used to induce multidrug resistance) increased the number of individual mitochondria in both cell lines (FIGS. 5A, 5B) and mitochondrial network disrupting nanoparticle (MIND NP) decreased the number of individual mitochondria in the MDA-MB-231 cells. Hypoxia increased the number of mitochondrial networks in both cell lines (FIGS. 5C, 5D) which may confer a survival advantage by placing mitochondria in anti-apoptotic conformations; mitochondrial network disrupting nanoparticle (MIND NP) treatment significantly reduced mitochondrial networks in both cell lines in normoxic and hypoxic conditions (FIGS. 5C, 5D). Hypoxia increased the mitochondrial footprint (total individual+networked mitochondria) (FIGS. 5E, 5F) while mitochondrial network disrupting nanoparticle (MIND NP) treatment significantly decreased the mitochondrial footprints in both cell lines. Analysis was normalized to area to account for differences in cell sizes. Mitochondrial network disrupting nanoparticles (MIND NPs) effectively fragment mitochondrial networks and change mitochondrial footprints in TNBC cells. Significance was determined using Welch's t-test in GraphPad Prism 9.0.

The mitochondrial network analysis (MiNA) demonstrates that there is more variability in the numbers of individual mitochondria in the MDA-MB-231 cells (FIG. 5A) than in the BT-549 cells (FIG. 5B). Five days of hypoxia significantly increased the number of individual mitochondria in both cell lines while only the hypoxic MDA-MB-231 cells treated with mitochondrial network disrupting nanoparticles (MIND NPs) showed a reduction in individual mitochondria. Five days of hypoxia significantly increased the number of mitochondrial networks per square micron in both the MDA-MB-231 cells (FIG. 5C) and the BT-549 cells (FIG. 5D).

Hypoxia increases the survival response of cells. Therefore, increased mitochondrial networks can benefit survival by conformationally minimizing access to pro-apoptotic binding sites on the outer mitochondrial membranes.

Treatment for two hours with 10 μM mitochondrial network disrupting nanoparticles (MIND NPs) significantly reduced mitochondrial networks in normoxic and hypoxic MDA-MB-231 cells (FIG. 5C) and BT-549 cells (FIG. 5D). Mitochondrial networks were not significantly altered for the controls (blank nanoparticles (NPs) with no peptide and anti-mitofusin2 peptide in solution) relative to untreated normoxic and hypoxic cells (FIGS. 50, 5D). Mitochondrial footprints represent the total combined networks and individual mitochondria. There was a similar pattern of hypoxic increase and mitochondrial network disrupting nanoparticle (MIND NP) decrease in mitochondrial footprints for both cell lines (FIG. 5E, 5F).

Inter- and intra-organelle fusion is central to contemporary cell biology. Increased mitochondrial network formation in multidrug resistant cancer may confer a unique survival advantage. Mitofusin 2 (MFN2), and to a lesser extent, mitofusin 1 (MFN1), mediates the fusion of mitochondria. When mitochondrial networks fiss apart, mitofusin 2 (MFN2) scission sites become recruiting sites for pro-apoptotic Bcl-2 family members (e.g., Bax corresponding to bcl-2-like protein 4) to bind to the outer mitochondrial membrane. Mitochondrial network formation creates a conformational resistance to such apoptotic signaling. Mitochondrial networks have a higher capacity for oxidative phosphorylation (OXPHOS), which also benefits multidrug resistant cancer cell survival. Mitochondrial networks are in constant flux in response to cellular demands, and the constant flux may explain the conflicting published data on oxidative phosphorylation (OXPHOS) changes in multidrug resistant cancer. As mitochondrial networks are continually fusing together and fissing apart, the cellular capacity for oxidative phosphorylation (OXPHOS) is also in flux. A third possible survival advantage that may be conferred by increased mitochondrial networks in multidrug resistant cancer cells is a paralleled increased in mitophagy and autophagy⁹⁴⁻¹⁰⁰. Mitochondrial recycling and repair (mitophagy) is critical to cellular health by removing reactive oxygen species (ROS) and damaged mitochondrial DNA⁹⁴⁻¹⁰⁰. Mitophagy can also trigger the larger process of autophagy, aiding cell survival⁹⁵. The present technology demonstrates that hypoxic induced multidrug resistance increases mitochondrial networks in triple negative breast cancer (TNBC) cells.

FIGS. 5A-F also demonstrate that mitochondrial network disrupting nanoparticles (MIND NPs) fragment mitochondrial networks in normoxic and hypoxic triple negative breast cancer (TNBC) cells. This fragmentation can be used to lower the apoptotic threshold in cancer cells and to sensitize cancer cells to subsequent stressors. Manipulation of mitochondrial dynamics represent untouched Achilles Heel of cancer therapeutics.

### Example 4: Combination Organelle Mitochondrial Endoplasmic Reticulum Therapy (COMET) Efficacy and Combination Index

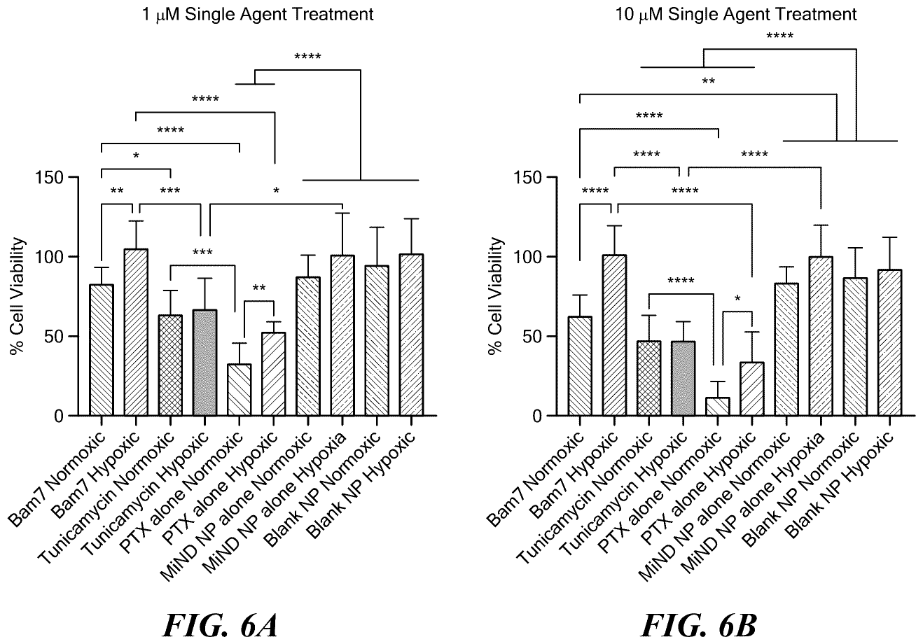

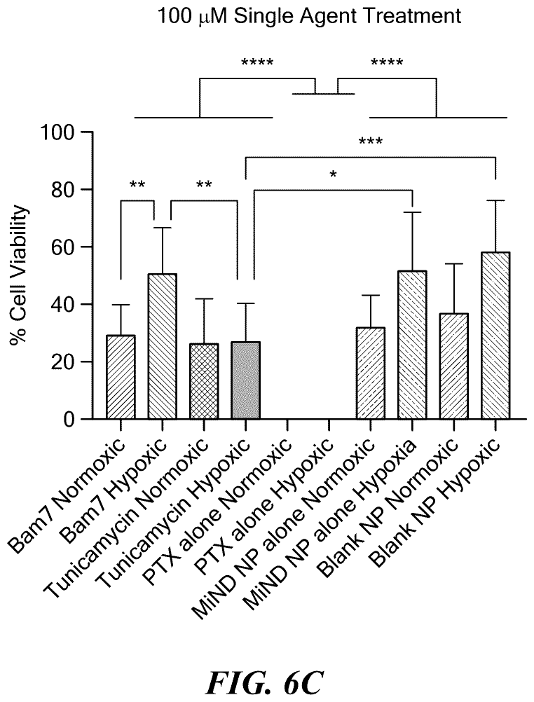

For investigating whether a combination organelle mitochondrial endoplasmic reticulum therapy (COMET) is effective at treating multidrug resistant cancer, a five day dose response study of the individual components of a combination organelle mitochondrial endoplasmic reticulum therapy (COMET) (0.01-100 μM), paclitaxel (PTX), and blank nanoparticle (NP) was conducted in normoxic and hypoxic MDA-MB-231 cells (FIGS. 6A-C). The half-maximal inhibitory concentration (IC₅₀) of the individual drug treatments and the ideal concentration of the combination treatment was determined. Single agent drug treatment at (FIG. 6A) 1 μM, (FIG. 6B) 10 μM, and (FIG. 6C) 100 μM in normoxic and hypoxic MDA-MB-231 cells progressed for five days. Cell viability was measured using Promega's 3-(4,5-dimethylthiazol-2-yl)-5-(3-carboxymethoxyphenyl)-2-(4-sulfophenyl)-2H-tetrazolium (MTS) assay and n=24. Tunicamycin is the only drug that has comparable efficacy in hypoxic and normoxic cells demonstrating that multidrug resistant cancer cells may not be resistant to tunicamycin. Conversely, hypoxic cells are more resistant to Bam7 which correlates to the anti-apoptotic conformation of increased mitochondrial networks. Treatment is compared to the standard of care treatment with paclitaxel (PTX). Significance was determined using Welch's t-test in GraphPad Prism 9.0.

FIGS. 6A-C show the results of monotherapy in hypoxic and normoxic MDA-MB-231 cells with the single drug treatment. The results of FIGS. 6A-C demonstrate that a combination organelle mitochondrial endoplasmic reticulum therapy (COMET) is as effective as treatment with paclitaxel (PTX) when treating drug sensitive and drug resistant triple negative breast cancer (TNBC) Cells. The agents of Bam7, Tunicamycin, and mitochondrial network disrupting nanoparticles (MIND NPs) demonstrated low efficacy at low doses (0.01 and 0.1 μM not shown) including 1 μM (FIG. 6A). Hypoxic cells were more resistant to all individual drug treatments except for tunicamycin. Interestingly, hypoxic/multidrug resistant cancer cells appear to be sensitized to tunicamycin as demonstrated by comparable efficacy for the normoxic and hypoxic conditions for all concentrations (FIGS. 6A-C). The increased resistance of hypoxic cells to Bam7 aligns with the increased mitochondrial networks seen in FIGS. 5A-F data as mitochondrial networks orient mitochondria in anti-apoptotic conformations and Bam7 is a pro-apoptotic Bax (bcl-2-like protein 4) activator. Blank nanoparticles had little effect on cell viability except at the very high concentration of 100 μM. Based on this study, the lowest drug concentrations for combination organelle mitochondrial endoplasmic reticulum therapy (COMET) were 10 μM MIND NPs, 1 μM Bam7, and 1 μM Tunicamycin.

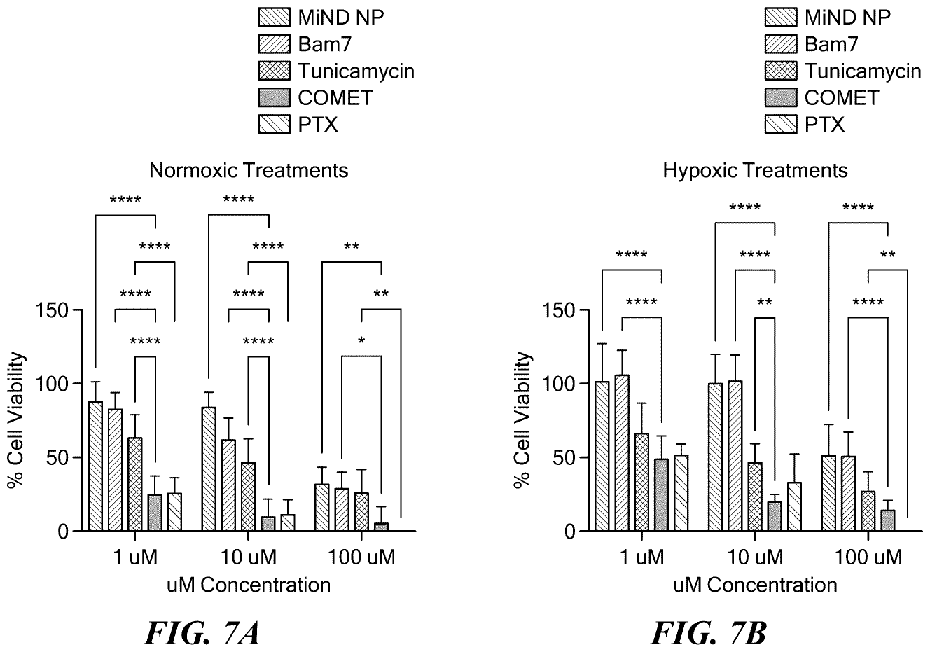

The rationale for a combination organelle mitochondrial endoplasmic reticulum therapy (COMET) is based on the successive exploitation of the mitochondrial-endoplasmic reticulum axis to induce cell death in multidrug resistant cancer tumor cells with less toxicity to healthy cells. For investigating whether the triple drug combination had a synergist combination index, normoxic (FIG. 7A) and hypoxic (FIG. 7B) cells were treated with mitochondrial network disrupting nanoparticles (MIND NPs); Bam7 (a direct autophagic inducer Bax activator); Tunicamycin (an endoplasmic reticulum stressor); a combination organelle mitochondrial-endoplasmic reticulum therapy (COMET) including triple combination of mitochondrial network disrupting nanoparticles (MIND NPs), Bam7, and tunicamycin; and Paclitaxel (PTX), a common chemotherapeutic agent). Treatment persisted for 5-days, and cell viability was measured using Promega's 3-(4,5-dimethylthiazol-2-yl)-5-(3-carboxymethoxyphenyl)-2-(4-sulfophenyl)-2H-tetrazolium (MTS) assay and n=24. Only intra-dose significance of combination organelle mitochondrial-endoplasmic reticulum therapy (COMET) is shown. The combination organelle mitochondrial-endoplasmic reticulum therapy (COMET) significantly improved the efficacy of all drugs in normoxic and hypoxic cells at 10 μM whereas paclitaxel (PTX) was not significantly different than tunicamycin at this concentration in hypoxic cells. Most notably, for all concentrations in both normoxic and hypoxic conditions there was no significant difference in combination organelle mitochondrial-endoplasmic reticulum therapy (COMET) treatment and treatment with standard of care paclitaxel (PTX). There was significance for all mitochondrial network disrupting nanoparticles (MIND NPs) and Bam7 relative to paclitaxel (PTX) for all concentrations and both conditions Significance was determined using Welch's t-test in (significance not shown). GraphPad Prism 9.0.

Data in FIGS. 6A-C and FIGS. 7A-B confirm that the mitochondrial network disrupting nanoparticles (MIND NPs) and Bam7 are ineffective as monotherapies. Tunicamycin is the only component of the combination organelle mitochondrial endoplasmic reticulum therapy (COMET) with appreciable efficacy as a monotherapy and interestingly, multidrug resistant triple negative breast cancer (TNBC) cells do not appear to demonstrate resistance to tunicamycin. For the study in FIGS. 7A-B, 1 μM Bam7 and 1 μM Tunicamycin were evaluated with different concentrations of mitochondrial network disrupting nanoparticles (MIND NPs). At 10 μM concentrations, the combination organelle mitochondrial endoplasmic reticulum therapy (COMET) was significantly more effective than tunicamycin alone under both normoxic and hypoxic conditions, and most importantly, under hypoxic conditions, there were no significant differences between tunicamycin and paclitaxel (PTX). At 100 μM, concentrations, there was no significance between tunicamycin and the combination organelle mitochondrial endoplasmic reticulum therapy (COMET), and yet there was a significant difference between tunicamycin and paclitaxel (PTX). The ideal concentration of mitochondrial network disrupting nanoparticles (MIND NPs) in this two-dimensional (2D) system is 10 μM which is also significantly more effective than 1 μM tunicamycin (not shown on graph). As mitochondrial network disrupting nanoparticles (MIND NPs) and Bam7 had very little efficacy as a monotherapy, further combination and safety analysis were explored.

For further assessment of whether treatment with mitochondrial network disrupting nanoparticles (MIND NPs) was effective in reducing the apoptotic threshold, a combination treatment with mitochondrial network disrupting nanoparticles (MIND NPs) and paclitaxel (PTX) was evaluated in normoxic and hypoxic cells.

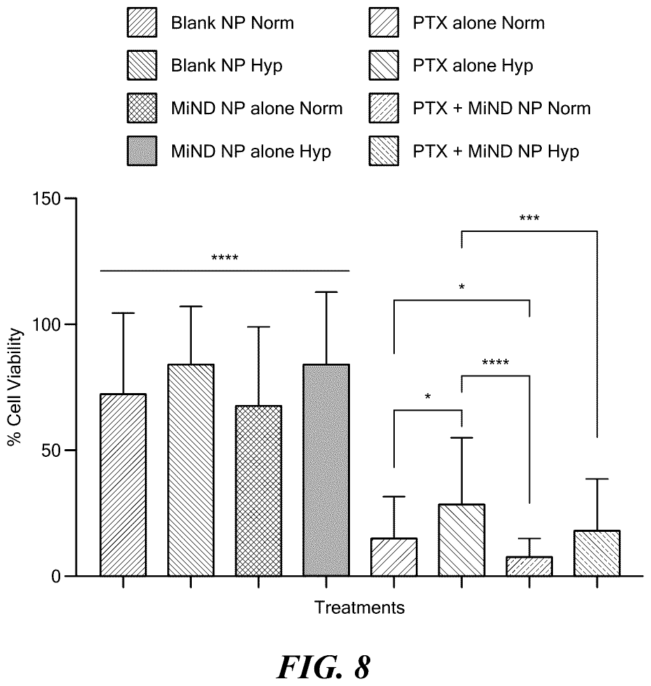

FIG. 8 shows the results of treatment with 10 μM mitochondrial network disrupting nanoparticles (MIND NPs) and 1 μM paclitaxel (PTX) progressed over 5 days. The effect of treatment with blank nanoparticles (NP), 10 μM mitochondrial network disrupting nanoparticles (MIND NPs), 1 μM paclitaxel (PTX), and combination paclitaxel (PTX) (1 μM) and mitochondrial network disrupting nanoparticles (MIND NPs) (10 μM) were evaluated in hypoxic (Hyp) multidrug resistant cancer and normoxic (Norm) MDA-MB-231 triple negative breast cancer (TNBC) cells after five days of treatment using Promega's MTS assay and n=24. Mitochondrial network disrupting nanoparticles (MIND NPs) were less toxic than paclitaxel (PTX) and, as a combination treatment, improved the efficacy of paclitaxel (PTX). The asterisks spanning blank nanoparticles and the and mitochondrial network disrupting nanoparticles (MIND NPs) alone indicate significance with paclitaxel (PTX) treatments and paclitaxel (PTX)/mitochondrial network disrupting nanoparticle (MIND NP) combination treatments. Significance was determined using Welch's t-test in GraphPad Prism 9.0.

FIG. 8 shows that mitochondrial network disrupting nanoparticle (MIND NP) monotherapy is safter than paclitaxel (PAX) monotherapy and mitochondrial network disrupting nanoparticles (MIND NPs) demonstrate efficacy in proof of concept therapy. Treatment with mitochondrial network disrupting nanoparticles (MIND NPs) significantly improved the efficacy of paclitaxel (PTX) in both normoxic and hypoxic conditions whereas mitochondrial network disrupting nanoparticles (MIND NPs) and blank nanoparticles (NPs) had little effect on cell viability but were significantly different than all other treatments.

The inhibitory concentration of a drug that kills 50% of the cell population (IC₅₀) is a useful parameter for comparing drug efficacy. Table 2 lists the IC₅₀ values for the monotherapies and combination treatments in normoxic and hypoxic MDA-MB-231 cells. Column three lists the fold change between normoxic and hypoxic conditions for the listed drug. Tunicamycin has the lowest fold change between normoxic and hypoxic IC₅₀'s and a low IC₅₀ value for both conditions (below 6 μM), validating that tunicamycin is a useful drug for treating multidrug resistant cancers such as multidrug resistant triple negative breast cancer (TNBC). Mitochondrial network disrupting nanoparticles (MIND NPs) and blank nanoparticles (NPs) demonstrated a 1.52 and 1.56 fold increase respectively and approached very high IC₅₀ concentrations (68-120 μM) demonstrating the lack of efficacy of these particles alone. The Bam7 agent had a similar profile. The fold change for paclitaxel (PTX) was a 70.67 increase in the IC₅₀ for hypoxic cells. This attests to paclitaxel (PTX) being a substrate for multidrug resistant 1 (MDR1) which is highly upregulated in multidrug resistant cancers such a multidrug resistant triple negative breast cancer (TNBC). This fold increase demonstrates the need for more effective standards of care that are not directly challenged by the biology of multidrug resistant cancer disease. Compared to paclitaxel (PTX), the fold increase for the hypoxic IC₅₀ was only 3.5 illustrating that a combination organelle mitochondrial endoplasmic reticulum therapy (COMET) may be more appropriate for treating multidrug resistant cancers such as multidrug resistant triple negative breast cancer (TNBC) than paclitaxel (PTX). The most dramatic fold increase in IC₅₀ values was for mitochondrial network disrupting nanoparticles (MIND NPs) in combination with paclitaxel (PTX); this increase was 3511.9. A possibility for this extreme difference may be that normoxic (drug sensitive) multidrug resistant cancers such as multidrug resistant triple negative breast cancer (TNBC) cells may be more susceptible to both pro-apoptotic conformational changes in mitochondrial networks and to paclitaxel (PTX) efficacy (lower multidrug resistant cancer expression). Table 2 demonstrates that a combination organelle mitochondrial endoplasmic reticulum therapy (COMET) improves the IC₅₀ of drugs and multidrug resistant cells are less resistant to a combination organelle mitochondrial endoplasmic reticulum therapy (COMET) than to paclitaxel (PTX).

TABLE 2
IC₅₀ values and Fold Changes for the Monotherapies and Combination
Treatments in Normoxic and Hypoxic MDA-MB-231 cells

| Treatment | IC₅₀ (μM) | Fold change |
|---|---|---|
| Bam7 Normoxic | 58.83 ± 20.97 | 1.73 |
| Bam7 Hypoxic | 101.52 ± 31.07 | |
| Tunicamycin Normoxic | 5.54 ± 1.65 | 1.07 |
| Tunicamycin Hypoxic | 5.92 ± 0.52 | |
| MiND NP Normoxic | 68.28 ± 13.67 | 1.52 |
| MiND NP Hypoxic | 104.02 ± 31.08 | |
| PTX Normoxic | 0.015 + 0.03 | 70.67 |
| PTX Hypoxic | 1.06 ± 0.31 | |
| COMET Normoxic | 0.22 ± 0.45 | 3.5 |
| COMET Hypoxic | 0.77 ± 1.10 | |
| PTX + MiND NP Normoxic | 0.000168 ± 0.0003 | 3511.9 |
| PTX + MiND NP Hypoxic | 0.59 ± 0.61 | |
| Blank NP Normoxic | 76.83 ± 4.30 | 1.56 |
| Blank NP Hypoxic | 119.58 ± 3.15 | |

The Combination Index (CI) and the Dose Reduction Index (DRI) were calculated for each drug combination in the cell panel¹⁰¹ and are listed in Table 3. The Cl is an indication of the value drug combinations; antagonism (over 1), additive effects (1), or synergism (less than 1)¹⁰¹. The DRI is an indication of the ability to reduce toxicity without reducing efficacy. The CI for mitochondrial network disrupting nanoparticles (MIND NPs) and paclitaxel (PTX) in both normoxic and hypoxic cells is synergistic in MDA-MB-231 cells, although much less synergistic (approaching closer to 1) for hypoxic cells. Similarly, there is a dramatically high DRI for mitochondrial network disrupting nanoparticles (MIND NPs) combined with paclitaxel (PTX). A possibility is that paclitaxel (PTX) (which hyperstablizes microtubules) is sensitizing normoxic cells to the proapoptotic conformational accessibility of fragmented mitochondria. The CI for combination organelle mitochondrial endoplasmic reticulum therapy (COMET) in both MDA-MB-231 cells and BT-549 cells is also synergistic with high DRI's for Bam7 and mitochondrial network disrupting nanoparticles (MIND NPs). The most important DRI for combination organelle mitochondrial endoplasmic reticulum therapy (COMET) in tunicamycin and this ranges from 7.69-25.96. Mitochondrial network disrupting nanoparticles (MIND NPs) and Bam7 have clear potential to lower the toxicity of tunicamycin. Table 3 shows that a combination organelle mitochondrial endoplasmic reticulum therapy (COMET) has a synergistic CI and high DRI.

TABLE 3
Combination Index (CI) and the Dose Reduction Index (DRI) for each
drug combination

| | | | DRI | DRI | DRI | DRI |
|---|---|---|---|---|---|---|
| Treatment | Cell Line | CI | Bam7 | Tun | PTX | MiND |
| PTX + MiND | MDA-MB-231 | 0.01 | — | — | 91.67 | 406,428.57 |
| NP Normoxic | | | | | | |
| PTX + MiND | MDA-MB-231 | 0.56 | — | — | 1.8 | 176.31 |
| NP Hypoxic | | | | | | |
| COMET | MDA-MB-231 | 0.05 | 244.68 | 25.18 | — | 310.36 |
| Normoxic | | | | | | |
| COMET | MDA-MB-231 | 0.15 | 131.84 | 7.69 | — | 135.09 |
| Hypoxic | | | | | | |
| COMET | BT-549 | 0.04 | 9459.26 | 22.96 | — | 2948.15 |
| Normoxic | | | | | | |
| COMET | BT-549 | 0.04 | 8552.63 | 25.56 | — | 1928.57 |
| Hypoxic | | | | | | |

### Example 5: Combination Organelle Mitochondrial Endoplasmic Reticulum Therapy (COMET) Toxicity

The clinical success of combination organelle mitochondrial endoplasmic reticulum therapy (COMET) would dramatically decrease overall drug exposure of patients suffering from multidrug resistance cancers such as multidrug resistant triple negative breast cancer (TNBC), thereby decreasing toxicity of cancer treatment. There is a critical clinical need for cancer treatments with lower toxicity.

In Example 5, the superior safety of COMET in Human Embryonic Kidney Epithelial (HEK) cells. Human embryonic kidney epithelial cells (293T) were treated with each drug for 48 hours and cell viability was determined using the MTS assay and n=24. Significance was determined using Welch's t-test in GraphPad Prism 9.0.

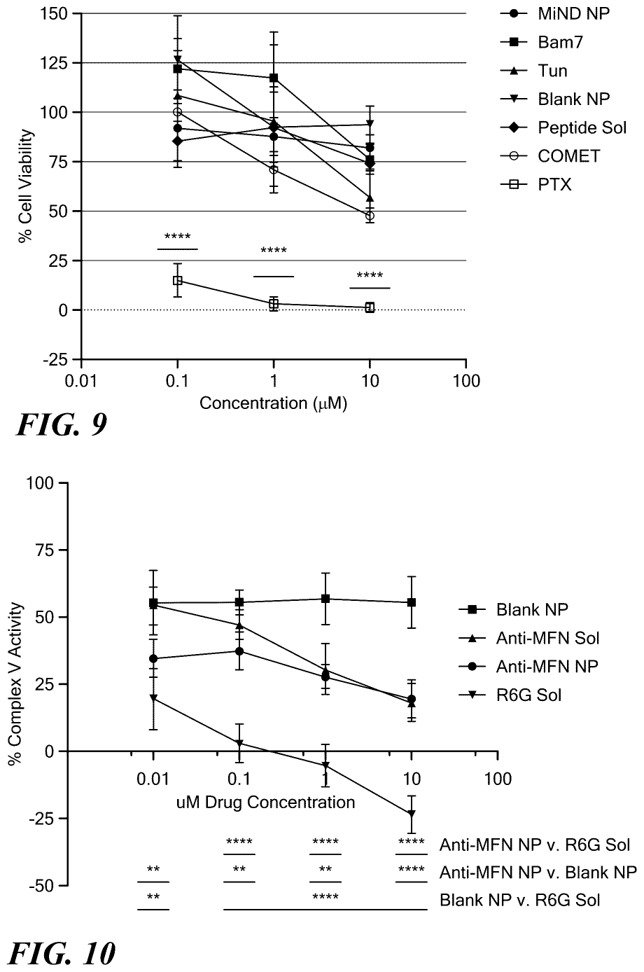

FIG. 9 shows that combination organelle mitochondrial endoplasmic reticulum therapy (COMET) is significantly safer and less toxic to normal cells than paclitaxel (PTX). There is a 2403 fold difference in the concentration of paclitaxel (PTX) as compared to the combination organelle mitochondrial endoplasmic reticulum therapy (COMET). Standard of care treatment with paclitaxel (PTX) is significantly more toxic than combination organelle mitochondrial endoplasmic reticulum therapy (COMET). The asterisk for each concentration depicts significance between paclitaxel (PTX) and all other treatments. As the dose of combination organelle mitochondrial endoplasmic reticulum therapy (COMET) increases on the log scale, the cell viability drops approximately 25% whereas the range for paclitaxel (PTX) is 25%-0% across the three concentrations. Combination organelle mitochondrial endoplasmic reticulum therapy (COMET) is an early phase translational nanomedicine with high potential for improved design and optimization. Minor improvements may allow for lower doses to achieve higher efficacy, resulting in dramatically improved safety for every log dose conserved. As is, without optimization, combination organelle mitochondrial endoplasmic reticulum therapy (COMET) is less toxic in HEK cells than paclitaxel (PTX).

### Example 6: Mitochondrial Network Disrupting Nanoparticle (MIND NP) Effect on Oxidative Phosphorylation (OXPHOS)

Mitochondria are multifaceted organelles. The previous examples have focused on evaluating the biological effect of mitochondrial network fragmentation by mitochondrial network disrupting nanoparticle (MIND NPs) in the context of the apoptotic threshold. The present technology hypothesizes lowering of the apoptotic threshold by mitochondrial network disrupting nanoparticle (MIND NPs) based on a combination of conformational accessibility of proapoptotic signaling to the outer mitochondrial membrane (not directly studied), de-coupling mitochondria from the endoplasmic reticulum, and decreasing the cellular energy capacity. This example is directed to exploring how the mitochondrial network disrupting nanoparticles (MIND NPs) affect the infamous powerhouse function of mitochondria.

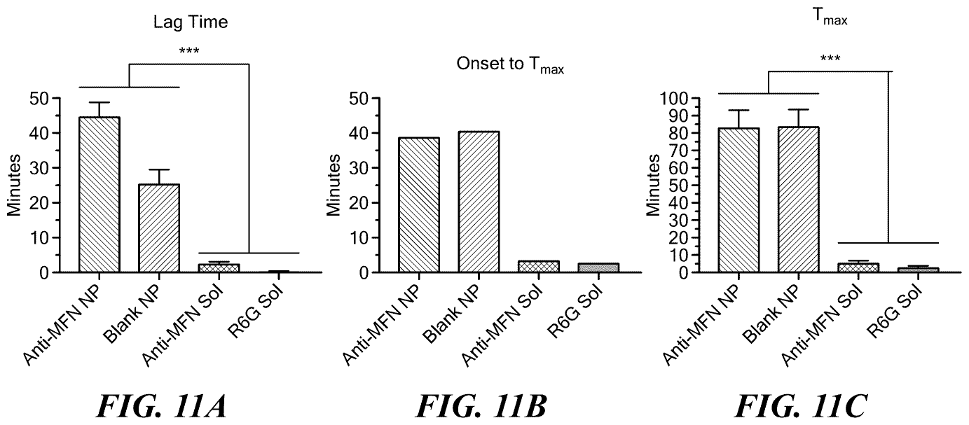

FIG. 10 depicts the effect of mitochondrial network disrupting nanoparticles (anti-mitofusin 2 nanoparticles (Anti-MFN2 NPs), anti-mitofusin 2 solution (Anti-MFN Sol), and blank nanoparticles (Blank NP) on complex V extracted from bovine mitochondria. Complex V is the adenosine triphosphate (ATP) producing subunit of the electron transfer chain. Rhodamine 6G (R6G Sol) is a specific complex V inhibitor and was used as a positive control. In FIG. 10, n=12. Significance was determined using Welch's t-test in GraphPad Prism 9.0.

After two hours of treatment, Abcam's MitoTox™ Complex V OXPHOS Assay demonstrated that both the mitochondrial network disrupting nanoparticles (Anti-MFN NP) and the anti-mitofusin 2 peptide solution (Anti-MFN Sol) significantly reduced and inhibited complex V activity relative to normal oxidative phosphorylation (OXPHOS) with no treatment and treatment with blank nanoparticles (Blank NP).

The mechanism of mitochondrial network disrupting nanoparticle (MIND NP) inhibition of complex V activity is unknown and may be explored in the future. In living cells, it is hypothesized that the mitochondrial network disrupting nanoparticle (MIND NPs) would reduce oxidative phosphorylation (OXPHOS) capacity due as fragmented mitochondria have a higher surface area (outer mitochondrial membrane) to internal volume (OXPHOS machinery) ratio, diluting the capacity for higher OXPHOS.

Although the exact mechanism is unknown, the kinetics of the reactions and Complex V inhibition were evaluated further as shown in FIGS. 11A-C. Abcam's MitoTox™ Complex V OXPHOS Assay quantified the lag time (time between treatment initiation and onset of action (FIG. 11A); the time from the onset of activity and Tmax (time of maximal activity) (FIG. 11B); and the Tmax (time of maximal activity) (FIG. 11C). Rhodamine 6 G (R6G Sol) was used as a positive control, and n=12. Significance was determined using Welch's t-test in GraphPad Prism 9.0.

FIG. 11A which shows the lag time between the initiation of treatment and the onset of activity demonstrates that the lag time associated with the mitochondrial network disrupting nanoparticles (Anti-MFN NP) likely was prolonged relative to the anti-mitofusin 2 peptide solution (Anti-MFN Sol) and the Rhodamine 6G solution (R6G Sol) because of the time for the anti-mitofusin 2 peptide to be released from the MIND NPs and the time for nanoparticle decomposition and possible lipid activity for the blank nanoparticles (Blank NP). The time from onset of activity to the time of maximal activity (Tₘₐₓ) (FIG. 10B) was obtained as an average and not analyzed for statistical significance. The Tₘₐₓ (FIG. 10C) was also prolonged for the nanoparticle formulations which aligns with solution drugs and lipids being more readily available than peptides encapsulated in nanoparticles and nanoparticle constituents breaking down.

### Example 7: Tunicamycin and the Unfolded Protein Response

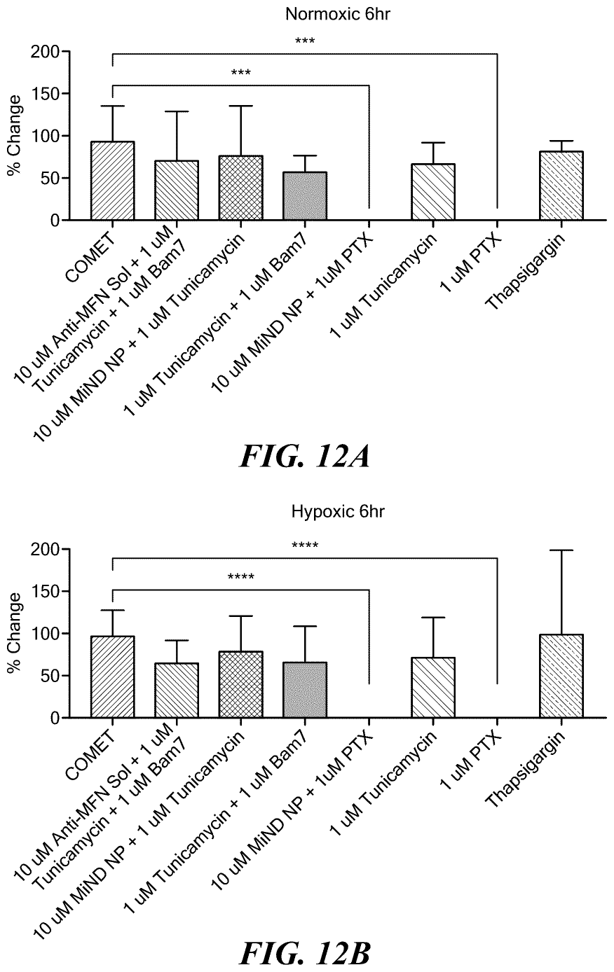

As a triple combination system, combination organelle mitochondrial endoplasmic reticulum therapy (COMET) has the potential to activate many levels of cell stress. Our rationale for including tunicamycin was to stress the endoplasmic reticulum and induce the unfolded protein response after robbing the ability of mitochondria to couple to the endoplasmic reticulum and rescue the endoplasmic reticulum from this stress. To evaluate if tunicamycin induces the unfolded protein response, an unfolded protein (UPR) endoplasmic reticulum stress assay was conducted in normoxic (FIG. 12A) and hypoxic (FIG. 12B) MDA-MB-231 cells after 6 hours. Assays at 12 hrs and 24 hours were also evaluated but data was not significantly different from the 6 hour experiment.

FIGS. 12A-B show that tunicamycin Induces the unfolded protein response (UPR). Montana Molecular UPR Assay was used to quantify endoplasmic reticulum (ER) stress and compared to the known unfolded protein response (UPR) inducer thapsigargin, Activity was evaluated after 6 hours of treatment in normoxic (FIG. 12A) and hypoxic (FIG. 12B) MDA-MB-231 cells. n=12. Significance is only shown for organelle mitochondrial endoplasmic reticulum therapy (COMET) and the paclitaxel (PTX) and organelle mitochondrial endoplasmic reticulum therapy (COMET) plus mitochondrial network disrupting nanoparticles (MIND NPs). There was no significance between organelle mitochondrial endoplasmic reticulum therapy (COMET) and all other treatments. Significance was determined using Welch's t-test in GraphPad Prism 9.0.

In FIGS. 12A-B, only significance between combination organelle mitochondrial endoplasmic reticulum therapy (COMET) and mitochondrial network disrupting nanoparticles (Mind NPs) plus paclitaxel (PTX) and combination organelle mitochondrial endoplasmic reticulum therapy (COMET) and paclitaxel (PTX) alone is shown. There was no significance between combination organelle mitochondrial endoplasmic reticulum therapy (COMET) and all other treatments, including the positive control, thapsigargin, that induces the unfolded protein response (UPR). Treatment included combination organelle mitochondrial endoplasmic reticulum therapy (COMET), the triple drug combination solution (10 M MIND NP, 1 μM Bam7, and 1 μM tunicamycin), tunicamycin and mitochondrial network disrupting nanoparticles (MIND NPs), tunicamycin and Bam7, mitochondrial network disrupting nanoparticles (Mind NPs) and paclitaxel (PTX), tunicamycin alone, paclitaxel (PTX) alone, and thapsigargin. As paclitaxel (PTX) hyperstablizes microtubules and mitochondrial network disrupting nanoparticles (MIND NPs) fragment mitochondrial networks, the unfolded protein response (UPR) was not induced by this combination or by paclitaxel (PTX) alone. Tunicamycin induced the unfolded protein response in the endoplasmic reticulum.

### Example 8: Bam7, Combination Organelle Mitochondrial Endoplasmic Reticulum Therapy (COMET), and Apoptosis

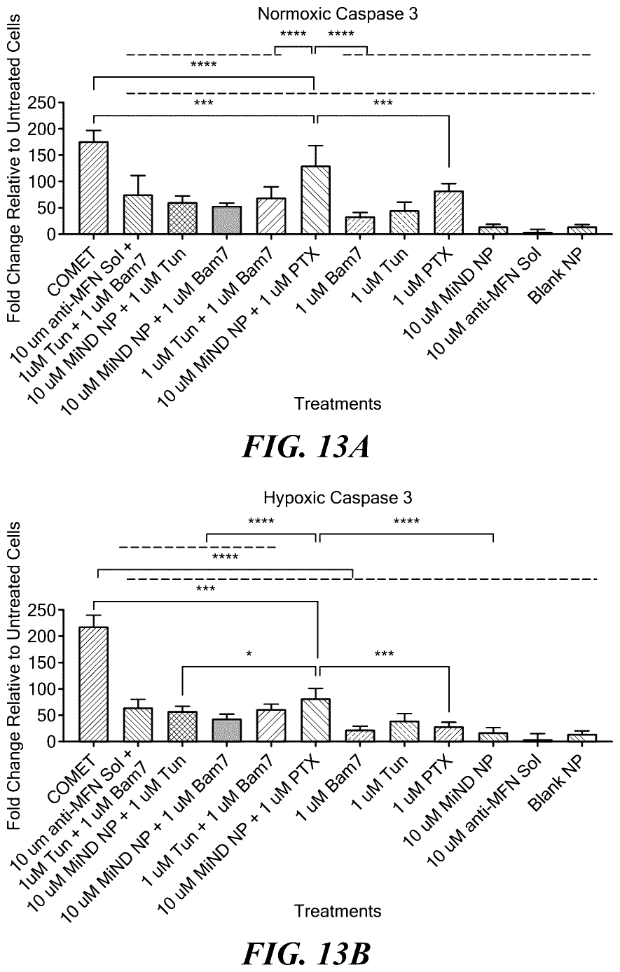

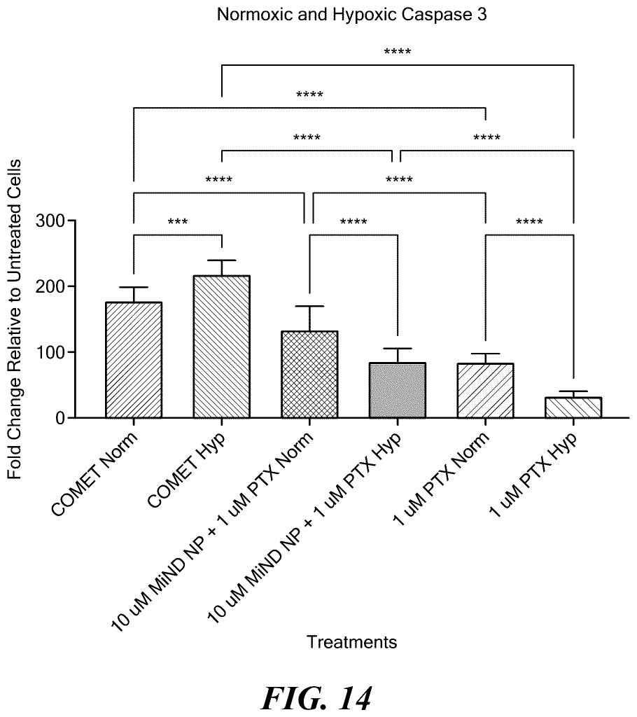

The third component of combination organelle mitochondrial endoplasmic reticulum therapy (COMET) is Bam7. Although Bam7 is a direct Bax (bcl-2-like protein 4) activator, it is a drug with weak activity, and it is not currently used clinically. The present technology includes Bam7 in combination organelle mitochondrial endoplasmic reticulum therapy (COMET) was to directly activate mitochondrial apoptosis after mitochondrial network fragmentation and after inducing endoplasmic reticulum stress. FIGS. 13A-B and FIG. 14 show the results of caspase 3 activity to explore the activity of Bam7 and combination organelle mitochondrial endoplasmic reticulum therapy (COMET) in inducing mitochondrial apoptosis. Caspase 3 is an effector caspase of both extrinsic (death receptor) and intrinsic (mitochondrial) apoptosis and is not specific to mitochondrial apoptosis.

Caspase 3 activity in normoxic (FIG. 13A) and hypoxic (FIG. 13B) MDA-MB-231 cells was evaluated using an n=14. Combination organelle mitochondrial endoplasmic reticulum therapy (COMET) significantly increased caspase 3 activity relative to all other treatments. The increase of caspase 3 after treatment with Bam7 alone at 1 μM was not comparable to combination organelle mitochondrial endoplasmic reticulum therapy (COMET). Significance was determined using Welch's t-test in GraphPad Prism 9.0.

Combination organelle mitochondrial endoplasmic reticulum therapy (COMET) increased the apoptotic activity of Bam7. Remarkably, combination organelle mitochondrial endoplasmic reticulum therapy (COMET) significantly increased caspase 3 activity relative to all other treatments in normoxic (FIG. 13A) and hypoxic normoxic (FIG. 13B) conditions. Bam7 alone (1 μM) or in combination with one other agent was insufficient at reaching comparable increases in caspase 3 activity relative to untreated cells. The triple combination of mitochondrial network disrupting nanoparticles (MIND NPs), 1 μM tunicamycin, and 1 μM Bam7 resulted in the most significant change even relative to paclitaxel (PTX) in combination with mitochondrial network disrupting nanoparticles (MIND NPs).

To more closely compare Combination organelle mitochondrial endoplasmic reticulum therapy (COMET) induction of caspase 3 in normoxic and hypoxic cells, data were graphed together in FIG. 14). FIG. 14 demonstrates the difference between hypoxic and normoxic caspase 3 induction. n=14. Significance was determined using Welch's t-test in GraphPad Prism 9.0.

Combination organelle mitochondrial endoplasmic reticulum therapy (COMET) resulted in a more significant increase in caspase 3 activity in hypoxic cells compared to normoxic cells treated with Combination organelle mitochondrial endoplasmic reticulum therapy (COMET). The significant increase in caspase 3 activity after Combination organelle mitochondrial endoplasmic reticulum therapy (COMET) treatment verses paclitaxel (PTX) treatment in hypoxic cells is promising as Combination organelle mitochondrial endoplasmic reticulum therapy (COMET) may be a better strategery for treating MDR TNBC than paclitaxel (PTX). Mitochondrial network disrupting nanoparticles (MIND NPs) combined with paclitaxel (PTX) also increased caspase 3 activity compared to paclitaxel (PTX) alone. This aligns with the Cl and DRI and the hypothesis of proapoptotic conformational sensitivity of mitochondria.

The present evaluation of combination organelle mitochondrial endoplasmic reticulum therapy (COMET) attests that mitochondria may be the untouched Achilles Heel of multidrug resistant cancer. Combination organelle mitochondrial endoplasmic reticulum therapy (COMET) disrupts mitochondrial networks with mitochondrial network disrupting nanoparticles (MIND NPs), induces the unfolded protein response (UPR) with tunicamycin, and induces mitochondrial apoptosis with Bam7. Combination organelle mitochondrial endoplasmic reticulum therapy (COMET) has demonstrated that successively fragmenting mitochondrial networks, stressing the endoplasmic reticulum, and inducing intrinsic apoptosis is a promising approach to treating multidrug resistant cancers such as multiple drug resistant triple negative breast cancer (MDR TNBC). Combination organelle mitochondrial endoplasmic reticulum therapy (COMET) has strong potential to reduce toxicity and as a safer molecular therapy than the current standard of care. Combination organelle mitochondrial endoplasmic reticulum therapy (COMET) could transform the treatment of multidrug resistant cancers such as triple negative breast cancer (MDR TNBC); improving clinical outcomes without dose-limiting toxicity.

### REFERENCES

1 Grandemange, S., Herzig, S. & Martinou, J.-C. Mitochondrial dynamics and cancer. Seminars in Cancer Biology 19, 50-56, doi: doi.org/10.1016/j.semcancer.2008.12.001 (2009).
2 Maycotte, P. et al. Mitochondrial dynamics and cancer. Tumor Biology 39, 1010428317698391, doi: 10.1177/1010428317698391 (2017).
3 Tikhomirova, M. S., Kadosh, A., Saukko-Paavola, A. J., Shemesh, T. & Klemm, R. W. A role for endoplasmic reticulum dynamics in the cellular distribution of microtubules. Proc Natl Acad Sci USA 119, e2104309119, doi: 10.1073/pnas.2104309119 (2022).
4 Li, C. et al. Endoplasmic Reticulum-Plasma Membrane Contact Sites: Regulators, Mechanisms, and Physiological Functions. Front Cell Dev Biol 9, 627700, doi: 10.3389/fcell.2021.627700 (2021).
5 Society, A. C. Breast Cancer Facts & Figures, <www.cancer.org/research/cancer-facts-statistics/breast-cancer-facts-figures.html> (2021).
6 Organization, W. H. Cancer Fact sheet, <www.who.int/news-room/fact-sheets/detail/cancer> (2022).
7 Bai, X., Ni, J., Beretov, J., Graham, P. & Li, Y. Triple-negative breast cancer therapeutic resistance: Where is the Achilles' heel? Cancer Letters 497, 100-111, doi: doi.org/10.1016/j.canlet.2020.10.016 (2021).
8 NCI. <www.cancer.gov/about-cancer/understanding/statistics> (9
9 NCI. Identifying Novel Drug Combinations to Overcome Treatment Resistance, <www.cancer.gov/about-cancer/treatment/research/drug-combo-resistance> (2016).
10 Milane, L. in Nanomedicine for Inflammatory Diseases (ed Mansoor Amiji and Lara Milane) Ch. Chapter 9, 319-331 (CRC Press, 2017).
11 Hanahan, D. & Weinberg, R. A. The Hallmarks of Cancer. Cell 100, 57-70, doi: doi.org/10.1016/S0092-8674 (00) 81683-9 (2000).
12 Hanahan, D. & Weinberg, Robert A. Hallmarks of Cancer: The Next Generation. Cell 144, 646-674, doi: 10.1016/j.cell.2011.02.013 (2011).
13 Gottesman, M. M., Fojo, T. & Bates, S. E. Multidrug resistance in cancer: role of ATP-dependent transporters. Nature Reviews Cancer 2, 48, doi: 10.1038/nrc706 (2002).
14 Housman, G. et al. Drug Resistance in Cancer: An Overview. Cancers 6, 1769-1792, doi: 10.3390/cancers6031769 (2014).
15 Juan-Carlos, P.-D. M., Perla-Lidia, P.-P., Stephanie-Talia, M.-M., Mónica-Griselda, A.-M. & Luz-María, T.-E. ABC transporter superfamily. An updated overview, relevance in cancer multidrug resistance and perspectives with personalized medicine. Molecular Biology Reports 48, 1883-1901, doi: 10.1007/s11033-021-06155-w (2021).
16 Vyas, S., Zaganjor, E. & Haigis, M. C. Mitochondria and Cancer. Cell 166, 555-566, doi: 10.1016/j.cell.2016.07.002 (2016).
17 Milane, L., Dolare, S., Jahan, T. & Amiji, M. Mitochondrial nanomedicine: Subcellular organelle-specific delivery of molecular medicines. Nanomedicine 37, 102422, doi: 10.1016/j.nano.2021.102422 (2021).
18 Filadi, R., Pendin, D. & Pizzo, P. Mitofusin 2: from functions to disease. Cell Death & Disease 9, 330, doi: 10.1038/s41419-017-0023-6 (2018).
19 Scorrano, L. Keeping mitochondria in shape: a matter of life and death. European Journal of Clinical Investigation 43, 886-893, doi: 10.1111/eci. 12135 (2013).
20 Franco, A. et al. Correcting mitochondrial fusion by manipulating mitofusin conformations. Nature 540, 74-79, doi: 10.1038/nature20156 (2016).
21 Landes, T. & Martinou, J.-C. Mitochondrial outer membrane permeabilization during apoptosis: The role of mitochondrial fission. Biochimica et Biophysica Acta (BBA)—Molecular Cell Research 1813, 540-545, doi: doi.org/10.1016/j.bbamcr.2011.01.021 (2011).
22 Renault, Thibaud T. et al. Mitochondrial Shape Governs BAX-Induced Membrane Permeabilization and Apoptosis. Molecular Cell 57, 69-82, doi: 10.1016/j.molcel.2014.10.028 (2015).
23 Chen, H., McCaffery, J. M. & Chan, D. C. Mitochondrial fusion protects against neurodegeneration in the cerebellum. Cell 130, 548-562, doi: 10.1016/j.cell.2007.06.026 (2007).
24 Yuan, H. et al. Mitochondrial fission is an upstream and required event for bax foci formation in response to nitric oxide in cortical neurons. Cell death and differentiation 14, 462-471, doi: 10.1038/sj.cdd.4402046 (2007).
25 Karbowski, M. et al. Spatial and temporal association of Bax with mitochondrial fission sites, Drp1, and Mfn2 during apoptosis. The Journal of cell biology 159, 931-938, doi: 10.1083/jcb.200209124 (2002).
26 Neuspiel, M., Zunino, R., Gangaraju, S., Rippstein, P. & McBride, H. Activated mitofusin 2 signals mitochondrial fusion, interferes with Bax activation, and reduces susceptibility to radical induced depolarization. The Journal of biological chemistry 280, 25060-25070, doi: 10.1074/jbc.M501599200 (2005).
27 Frank, S. et al. The role of dynamin-related protein 1, a mediator of mitochondrial fission, in apoptosis. Developmental cell 1, 515-525, doi: 10.1016/s1534-5807 (01) 00055-7 (2001).
28 Martineau, Y., Müller, D. & Pyronnet, S. Targeting protein synthesis in cancer cells. Oncoscience 1, 484-485, doi: 10.18632/oncoscience.63 (2014).
29 Lindqvist, L. M., Tandoc, K., Topisirovic, I. & Furic, L. Cross-talk between protein synthesis, energy metabolism and autophagy in cancer. Current Opinion in Genetics & Development 48, 104-111, doi: doi.org/10.1016/j.gde.2017.11.003 (2018).
30 Pervin, S. et al. Increased Susceptibility of Breast Cancer Cells to Stress Mediated Inhibition of Protein Synthesis. Cancer Research 68, 4862-4874, doi: 10.1158/0008-5472.can-08-0074 (2008).
31 Bravo-Sagua, R. et al. Cell death and survival through the endoplasmic reticulum-mitochondrial axis. Curr Mol Med 13, 317-329, doi: 10.2174/156652413804810781 (2013).
32 de Brito, O. M. & Scorrano, L. Mitofusin 2 tethers endoplasmic reticulum to mitochondria. Nature 456, 605, doi: 10.1038/nature07534 www.nature.com/articles/nature07534 #supplementary-information (2008).
33 Martinvalet, D. The role of the mitochondria and the endoplasmic reticulum contact sites in the development of the immune responses. Cell Death & Disease 9, 336, doi: 10.1038/s41419-017-0237-7 (2018).
34 Naon, D. et al. Critical reappraisal confirms that Mitofusin 2 is an endoplasmic reticulum-mitochondria tether. Proceedings of the National Academy of Sciences 113, 11249 (2016).
35 Williamson, C. D., Wong, D. S., Bozidis, P., Zhang, A. & Colberg-Poley, A. M. Isolation of Endoplasmic Reticulum, Mitochondria, and Mitochondria-Associated Membrane and Detergent Resistant Membrane Fractions from Transfected Cells and from Human Cytomegalovirus-Infected Primary Fibroblasts. Current protocols in cell biology/editorial board, Juan S. Bonifacino . . . [et al.] 68, 3.27.21-23.27.33, doi: 10.1002/0471143030.cb0327s68 (2015).
36 Bravo, R. et al. Endoplasmic Reticulum and the Unfolded Protein Response: Dynamics and Metabolic Integration. International review of cell and molecular biology 301, 215-290, doi: 10.1016/B978-0-12-407704-1.00005-1 (2013).
37 McGrath, E. P. et al. The Unfolded Protein Response in Breast Cancer. Cancers 10, 344 (2018).
38 Oslowski, C. M. & Urano, F. Measuring endoplasmic reticulum stress and the unfolded protein response using mammalian tissue culture system. Methods in enzymology 490, 71-92, doi: 10.1016/B978-0-12-385114-7.00004-0 (2011).
39 Peñaranda Fajardo, N. M., Meijer, C. & Kruyt, F. A. E. The endoplasmic reticulum stress/unfolded protein response in gliomagenesis, tumor progression and as a therapeutic target in glioblastoma. Biochemical Pharmacology 118, 1-8, doi: doi.org/10.1016/j.bcp.2016.04.008 (2016).
40 Milisav, I., Šuput, D. & Ribarič, S. Unfolded Protein Response and Macroautophagy in Alzheimer's, Parkinson's and Prion Diseases. Molecules 20, doi: 10.3390/molecules201219865 (2015).
41 Bravo, R. et al. Increased endoplasmic reticulum-mitochondrial coupling promotes mitochondrial respiration and bioenergetics during early phases of endoplasmic reticulum stress. Journal of Cell Science 124, 2143-2152, doi: 10.1242/jcs.080762 (2011).
42 Bravo, R. et al. in International Review of Cell and Molecular Biology Vol. 301 (ed Kwang W. Jeon) 215-290 (Academic Press, 2013).
43 da Silva, D. C., Valentão, P., Andrade, P. B. & Pereira, D. M. Endoplasmic reticulum stress signaling in cancer and neurodegenerative disorders: Tools and strategies to understand its complexity. Pharmacological Research 155, 104702, doi: doi.org/10.1016/j.phrs.2020.104702 (2020).
44 Gavathiotis, E., Reyna, D. E., Bellairs, J. A., Leshchiner, E. S. & Walensky, L. D. Direct and selective small-molecule activation of proapoptotic BAX. Nature chemical biology 8, 639-645, doi: 10.1038/nchembio.995 (2012).
45 Lee, J. S., Yost, S. E. & Yuan, Y. Neoadjuvant Treatment for Triple Negative Breast Cancer: Recent Progresses and Challenges. Cancers (Basel) 12, doi: 10.3390/cancers12061404 (2020).
46 Wahba, H. A. & El-Hadaad, H. A. Current approaches in treatment of triple-negative breast cancer. Cancer Biol Med 12, 106-116, doi: 10.7497/j.issn.2095-3941.2015.0030 (2015).
47 Irvine, D. J. & Dane, E. L. Enhancing cancer immunotherapy with nanomedicine. Nature reviews. Immunology 20, 321-334, doi: 10.1038/s41577-019-0269-6 (2020).
48 Donahue, N. D., Acar, H. & Wilhelm, S. Concepts of nanoparticle cellular uptake, intracellular trafficking, and kinetics in nanomedicine. Adv Drug Deliv Rev 143, 68-96, doi: 10.1016/j.addr.2019.04.008 (2019).
49 Milane, L. & Amiji, M. Clinical approval of nanotechnology-based SARS-COV-2 mRNA vaccines: impact on translational nanomedicine. Drug Deliv Transl Res, doi: 10.1007/s13346-021-00911-y (2021).
50 Milane, L., Trivedi, M., Singh, A., Talekar, M. & Amiji, M. Mitochondrial biology, targets, and drug delivery. J Control Release 207, 40-58, doi: 10.1016/j.jconrel.2015.03.036 (2015).
51 Susa, M., Milane, L., Amiji, M. M., Hornicek, F. J. & Duan, Z. Nanoparticles: a promising modality in the treatment of sarcomas. Pharm Res 28, 260-272, doi: 10.1007/s11095-010-0173-z (2011).
52 Shi, Y., van der Meel, R., Chen, X. & Lammers, T. The EPR effect and beyond: Strategies to improve tumor targeting and cancer nanomedicine treatment efficacy. Theranostics 10, 7921-7924, doi: 10.7150/thno.49577 (2020).
53 Corbett, K. S. et al. SARS-COV-2 mRNA Vaccine Development Enabled by Prototype Pathogen Preparedness. bioRxiv, doi: 10.1101/2020.06.11.145920 (2020).
54 Corbett, K. S. et al. Evaluation of the mRNA-1273 Vaccine against SARS-CoV-2 in Nonhuman Primates. N Engl J Med, doi: 10.1056/NEJMoa2024671 (2020).
55 Dong, Y. et al. A systematic review of SARS-COV-2 vaccine candidates. Signal Transduction and Targeted Therapy 5, 237, doi: 10.1038/s41392-020-00352-y (2020).
56 Fuller, D. H. & Berglund, P. Amplifying RNA Vaccine Development. N Engl J Med 382, 2469-2471, doi: 10.1056/NEJMcibr2009737 (2020).
57 Han, H. J., Nwagwu, C., Anyim, O., Ekweremadu, C. & Kim, S. COVID-19 and cancer: From basic mechanisms to vaccine development using nanotechnology. International immunopharmacology 90, 107247, doi: 10.1016/j.intimp.2020.107247 (2021).
58 Huang, L. et al. SARS-COV-2 vaccine research and development: Conventional vaccines and biomimetic nanotechnology strategies. Asian Journal of Pharmaceutical Sciences, doi: doi.org/10.1016/j.ajps.2020.08.001 (2020).
59 Keech, C. et al. Phase 1-2 Trial of a SARS-COV-2 Recombinant Spike Protein Nanoparticle Vaccine. New England Journal of Medicine 383, 2320-2332, doi: 10.1056/NEJMoa2026920 (2020).
60 Kim, J. H., Marks, F. & Clemens, J. D. Looking beyond COVID-19 vaccine phase 3 trials. Nat Med 27, 205-211, doi: 10.1038/s41591-021-01230-y (2021).
61 Lundstrom, K. The Current Status of COVID-19 Vaccines. Frontiers in Genome Editing 2, doi: 10.3389/fgeed.2020.579297 (2020).
62 Wolfram, J. & Ferrari, M. Clinical Cancer Nanomedicine. Nano today 25, 85-98, doi: 10.1016/j.nantod.2019.02.005 (2019).
63 Fang, J., Islam, W. & Maeda, H. Exploiting the dynamics of the EPR effect and strategies to improve the therapeutic effects of nanomedicines by using EPR effect enhancers. Adv Drug Deliv Rev 157, 142-160, doi: 10.1016/j.addr.2020.06.005 (2020).
64 Wu, J. The Enhanced Permeability and Retention (EPR) Effect: The Significance of the Concept and Methods to Enhance Its Application. Journal of personalized medicine 11, doi: 10.3390/jpm11080771 (2021).
65 Network, N. C. C. (2021).
66 Pawar, G. N., Parayath, N. N., Nocera, A. L., Bleier, B. S. & Amiji, M. M. Direct CNS delivery of proteins using thermosensitive liposome-in-gel carrier by heterotopic mucosal engrafting. PLOS ONE 13, e0208122, doi: 10.1371/journal.pone.0208122 (2018).
67 Valente, A. J., Maddalena, L. A., Robb, E. L., Moradi, F. & Stuart, J. A. A simple ImageJ macro tool for analyzing mitochondrial network morphology in mammalian cell culture. Acta Histochem 119, 315-326, doi: 10.1016/j.acthis.2017.03.001 (2017).
68 Brinkmann, J. et al. About supramolecular systems for dynamically probing cells. Chemical Society Reviews 43, 4449-4469, doi: 10.1039/C4CS00034J (2014).
69 Gao, S. & Hu, J. Mitochondrial Fusion: The Machineries In and Out. Trends in Cell Biology 31, 62-74, doi: 10.1016/j.tcb.2020.09.008 (2021).
70 Daste, F. et al. The heptad repeat domain 1 of Mitofusin has membrane destabilization function in mitochondrial fusion. EMBO reports 19, e43637, doi: doi.org/10.15252/embr.201643637 (2018).
71 Zacharioudakis, E. et al. Direct Small Molecule Activation of Mitofusins. bioRxiv, 301713, doi: 10.1101/301713 (2018).
72 Comerford, K. M. et al. Hypoxia-inducible Factor-1-dependent Regulation of the Multidrug Resistance (&lt;strong&gt;&lt;em&gt; multidrug resistant 1&lt;/em&gt;&lt;/strong&gt;) Gene. Cancer Research 62, 3387 (2002).
73 Harris, A. L. Hypoxia—a key regulatory factor in tumour gFigureth. Nature Reviews Cancer 2, 38-47, doi: 10.1038/nrc704 (2002).
74 Kurtoglu, M. & Lampidis, T. J. From delocalized lipophilic cations to hypoxia: blocking tumor cell mitochondrial function leads to therapeutic gain with glycolytic inhibitors. Mol Nutr Food Res 53, 68-75, doi: 10.1002/mnfr.200700457 (2009).
75 Li, D. W. et al. Hypoxia induced multidrug resistance of laryngeal cancer cells via hypoxia-inducible factor-1α. Asian Pacific journal of cancer prevention: APJCP 14, 4853-4858, doi: 10.7314/apjcp.2013.14.8.4853 (2013).
76 Milane, L., Duan, Z. & Amiji, M. Role of hypoxia and glycolysis in the development of multi-drug resistance in human tumor cells and the establishment of an orthotopic multi-drug resistant tumor model in nude mice using hypoxic pre-conditioning. Cancer Cell Int 11, 3, doi: 10.1186/1475-2867-11-3 (2011).
77 Milane, L., Ganesh, S., Shah, S., Duan, Z. F. & Amiji, M. Multi-modal strategies for overcoming tumor drug resistance: hypoxia, the Warburg effect, stem cells, and multifunctional nanotechnology. J Control Release 155, 237-247, doi: 10.1016/j.jconrel.2011.03.032 (2011).
78 Semenza, G. L. Pharmacologic Targeting of Hypoxia-Inducible Factors. Annual review of pharmacology and toxicology 59, 379-403, doi: 10.1146/annurev-pharmtox-010818-021637 (2019).
79 Zhang, H. et al. Krüppel-like factor 8 contributes to hypoxia-induced multidrug resistant cancer in gastric cancer cells. Cancer science 105, 1109-1115, doi: 10.1111/cas.12483 (2014).
80 Jing, X. et al. Role of hypoxia in cancer therapy by regulating the tumor microenvironment. Mol Cancer 18, 157, doi: 10.1186/s12943-019-1089-9 (2019).
81 Sebestyen, A., Kopper, L., Danko, T. & Timar, J. Hypoxia Signaling in Cancer: From Basics to Clinical Practice. Pathol Oncol Res 27, 1609802, doi: 10.3389/pore.2021.1609802 (2021).
82 López-Lázaro, M. The warburg effect: why and how do cancer cells activate glycolysis in the presence of oxygen? Anti-cancer agents in medicinal chemistry 8, 305-312, doi: 10.2174/187152008783961932 (2008).
83 Vander Heiden, M. G., Cantley, L. C. & Thompson, C. B. Understanding the Warburg effect: the metabolic requirements of cell proliferation. Science (New York, N.Y.) 324, 1029-1033, doi: 10.1126/science. 1160809 (2009).
84 Wicks, E. E. & Semenza, G. L. Hypoxia-inducible factors: cancer progression and clinical translation. J Clin Invest 132, doi: 10.1172/JCI159839 (2022).
85 Milane, L., Duan, Z. F. & Amiji, M. Pharmacokinetics and biodistribution of lonidamine/paclitaxel loaded, EGFR-targeted nanoparticles in an orthotopic animal model of multi-drug resistant breast cancer. Nanomedicine 7, 435-444, doi: 10.1016/j.nano.2010.12.009 (2011).
86 Milane, L., Duan, Z. & Amiji, M. Development of EGFR-targeted polymer blend nanocarriers for combination paclitaxel/lonidamine delivery to treat multi-drug resistance in human breast and ovarian tumor cells. Mol Pharm 8, 185-203, doi: 10.1021/mp1002653 (2011).
87 Milane, L., Duan, Z. & Amiji, M. Therapeutic efficacy and safety of paclitaxel/lonidamine loaded EGFR-targeted nanoparticles for the treatment of multi-drug resistant cancer. PLOS One 6, e24075, doi: 10.1371/journal.pone.0024075 (2011).
88 LazaFigureski, A. J. et al. Cardiomyocytes of chronically ischemic pig hearts express the multidrug resistant cancer-1 gene-encoded P-glycoprotein. The journal of histochemistry and cytochemistry: official journal of the Histochemistry Society 53, 845-850, doi: 10.1369/jhc.4A6542.2005 (2005).
89 LazaFigureski, A. et al. Neuronal mdr-1 gene expression after experimental focal hypoxia: a new obstacle for neuroprotection? Journal of the neurological sciences 258, 84-92, doi: 10.1016/j.jns.2007.03.004 (2007).
90 Erin, N., Grahovac, J., Brozovic, A. & Efferth, T. Tumor microenvironment and epithelial mesenchymal transition as targets to overcome tumor multidrug resistance. Drug resistance updates: reviews and commentaries in antimicrobial and anticancer chemotherapy 53, 100715, doi: 10.1016/j.drup.2020.100715 (2020).
91 Xie, Y. & Zhong, D. W. AEG-1 is associated with hypoxia-induced hepatocellular carcinoma chemoresistance via regulating PI3K/AKT/HIF-1 alpha/multidrug resistant cancer-1 pathway. EXCLI journal 15, 745-757, doi: 10.17179/excli2016-694 (2016).
92 Shome, R. & Ghosh, S. S. Transferrin Coated d-penicillamine-Au—Cu Nanocluster PLGA Nanocomposite Reverses Hypoxia-Induced EMT and multidrug resistant cancer of Triple-Negative Breast Cancers. ACS applied bio materials 4, 5033-5048, doi: 10.1021/acsabm. 1c00296 (2021).
93 Koshiba, T. et al. Structural basis of mitochondrial tethering by mitofusin complexes. Science 305, 858-862, doi: 10.1126/science. 1099793 (2004).
94 Chan, N. C. et al. Broad activation of the ubiquitin-proteasome system by Parkin is critical for mitophagy. Human Molecular Genetics 20, 1726-1737, doi: 10.1093/hmg/ddr048 (2011).
95 Ferro, F. et al. Autophagy and mitophagy in cancer metabolic remodelling. Seminars in Cell & Developmental Biology 98, 129-138, doi: doi.org/10.1016/j.semcdb.2019.05.029 (2020).
96 Guan, Y. et al. Mitophagy in carcinogenesis, drug resistance and anticancer therapeutics. Cancer Cell International 21, 350, doi: 10.1186/s12935-021-02065-w (2021).
97 Matsuda, N. et al. PINK1 stabilized by mitochondrial depolarization recruits Parkin to damaged mitochondria and activates latent Parkin for mitophagy. Journal of Cell Biology 189, 211-221, doi: 10.1083/jcb.200910140 (2010).
98 Mclelland, G.-L. et al. Mfn2 ubiquitination by PINK1/parkin gates the p97-dependent release of endoplasmic reticulum from mitochondria to drive mitophagy. eLife 7, e32866, doi: 10.7554/eLife.32866 (2018).
99 Xiao, L. et al. The mitochondria-targeted antioxidant MitoQ ameliorated tubular injury mediated by mitophagy in diabetic kidney disease via Nrf2/PINK1. Redox Biology 11, 297-311, doi: doi.org/10.1016/j.redox.2016.12.022 (2017).
100 Zhang, J. & Ney, P. A. Role of BNIP3 and NIX in cell death, autophagy, and mitophagy. Cell Death & Differentiation 16, 939-946, doi: 10.1038/cdd.2009.16 (2009).
101 Chou, T.-C. Drug Combination Studies and Their Synergy Quantification Using the Chou-Talalay Method. Cancer Research 70, 440-446, doi: 10.1158/0008-5472.can-09-1947 (2010).

1. A composition for treatment of a multidrug resistant cancer in a mammal, the composition comprising one or more of:
a mitochondrial fragmentation agent;
an unfolded protein response enhancing agent; and
an apoptosis activating agent.

2. The composition of claim 1, wherein the composition comprises a mitochondrial fragmentation agent which is an anti-mitofusin peptide.

3. The composition of claim 2, wherein the anti-mitofusin peptide is an anti-mitofusin 2 peptide comprising SEQ ID NO:1 (QDRLKFIDKQGELLAQDYKLR), or a fragment or variant thereof having at least 80% identity to SEQ ID NO: 1.

4. The composition of claim 1, wherein the composition comprises an unfolded protein response enhancing agent which is tunicamycin.

5. The composition of claim 1, wherein the composition comprises an apoptosis activating agent which is Bam7.

6. The composition of claim 1, wherein one or more of said agents are packaged in a plurality of lipid nanoparticles or polymeric nanoparticles.

7. The composition of claim 6, wherein the composition comprises a plurality of lipid nanoparticles which are cationic liposomes.

8. The composition of claim 7, wherein the cationic liposomes comprise cationic lipid 1,2-dioleoyl-3-trimethylammonium-propane (DOTAP) and further comprise one or more components selected from the group consisting of neutral phospholipids and cholesterol, and wherein one or more components of the liposomes is optionally conjugated to a hydrophilic polymer such as polyethylene glycol (PEG).

9. The composition of claim 7, wherein the cationic liposomes have a zeta potential greater than about +30 mV or less than about −30 mV.

10. The composition of claim 6, wherein the composition comprises a plurality of polymeric nanoparticles which are biodegradable polymeric nanoparticles.

11. The composition of claim 10, wherein the biodegradable polymeric nanoparticles comprise one or more biodegradable polymeric polymers selected from the group consisting of gelatin, chitosan, cellulose, cellulose derivatives, poly(ε-caprolactone) (PCL), polyethylene glycol (PEG), poly(lactic-co-glycolic acid) (PLGA), polylactic acid (PLA), polyglycolide, PEG-PLA diblock copolymer, PEG-PLGA diblock copolymer, PEG-PCL diblock copolymer, PCL-b-PEG-b-PCL co-polymer, polypropylene glycol (PPG), polyacrylic acid, polyacrylamide, poly(N-isopropylacrylamide), polyethylene oxide-polypropylene oxide-polyethylene oxide (PEO-PPO-PEO) triblock copolymer, hyaluronic acid, polyethylene oxide, polypropylene oxide, alginic acid, poly(beta-aminoester) (PBAE), poly(glycolic acid) (PGA), and alginate.

12. The composition of claim 6, wherein at least one of said agents is packaged in the lipid or polymeric nanoparticles, and at least one of said agents is not packaged in the lipid or polymeric nanoparticles.

13. The composition of claim 12, wherein the at least one of said agents that is not packaged in the lipid or polymeric nanoparticles is disposed in a liquid phase in which the lipid or polymeric nanoparticles are suspended.

14. The composition of claim 6, wherein the nanoparticles are cationic liposomes and at least one of said agents is disposed within a lipid bilayer phase of the liposomes or within a lumen of the liposomes.

15. The composition of claim 14, wherein the cationic liposomes comprise said mitochondrial fragmentation agent disposed within the lumen of the liposomes, and/or wherein the cationic liposomes comprise said unfolded protein response enhancing agent disposed within the lipid bilayer phase of the liposomes, and/or wherein the cationic liposomes comprise said apoptosis activating agent within the lipid bilayer phase of the liposomes.

16. The composition of claim 15, wherein the cationic liposomes comprise an anti-mitofusin 2 peptide disposed within the lumen of the liposomes, and comprise tunicamycin and Bam7 disposed within the lipid bilayer phase of the liposomes.

17. (canceled)

18. The composition of claim 1, wherein the one or more active agents are packaged into a plurality of lipid nanoparticles or polymeric nanoparticles, and wherein the lipid nanoparticles or polymeric nanoparticles comprise a targeting agent that promotes selective delivery of said active agents to targeted cancer cells.

19.-20. (canceled)

21. A method of treating a multidrug resistant cancer in a mammalian subject in need thereof, the method comprising:
(a) providing the composition of claim 1; and
(b) administering the composition to the mammalian subject, whereby death of multidrug resistant cancer cells of the mammalian subject is increased.

22. The method of claim 21, wherein, in cancer cells of the mammalian subject, fragmentation of mitochondria or a mitochondrial-endoplasmic reticulum network is enhanced, and/or wherein protein misfolding is enhanced, and/or wherein apoptosis is enhanced.

23.-25. (canceled)

26. A kit comprising the composition of claim 1 and one or more additional components, such as one or more additional therapeutic agents for treatment of a cancer and/or instructions for carrying out a method of treating a multidrug resistant cancer in a subject in need thereof, the method comprising:
(a) providing the composition of claim 1; and
(b) administering the composition to the mammalian subject, whereby death of multidrug resistant cancer cells of the mammalian subject is increased.
